(19) United States
(12) Patent Application Publication
(10) Pub. No.: US 2026/0158169 A1
(43) Pub. Date: Jun. 11, 2026

(54) OPTIMIZED SLC52A2 POLYNUCLEOTIDE CONSTRUCTS AND EXPRESSION CASSETTES

(71) Applicant: THE BOARD OF REGENTS OF THE UNIVERSITY OF TEXAS SYSTEM, Austin, TX (US)

(72) Inventors: Steven J. Gray, Southlake, TX (US); Xin Chen, Dallas, TX (US)

(73) Assignee: THE BOARD OF REGENTS OF THE UNIVERSITY OF TEXAS SYSTEM, Austin, TX (US)

(21) Appl. No.: 19/411,004

(22) Filed: Dec. 5, 2025

Related U.S. Application Data
(60) Provisional application No. 63/729,243, filed on Dec. 6, 2024.
(60) Provisional application No. 63/761,068, filed on Feb. 20, 2025.

(51) Int. Cl. A61K 48/00; A61K 38/17; A61P 25/28; C12N 15/86

(52) U.S. Cl. CPC A61K 48/005; A61K 38/1787; A61P 25/28; C12N 15/86; C12N 2750/14143; C12N 2830/50

(57) ABSTRACT

Provided herein are polynucleotide constructs comprising codon-optimized SLC52A2 gene, vectors (viral or non-viral vectors) comprising the polynucleotide constructs, methods of using the polynucleotide constructs for delivery of an SLC52A2 polynucleotide construct to a cell or a subject, and methods of using the polynucleotide constructs for treating SLC52A2 deficiency such as Riboflavin Transporter Deficiency (RTD).

### CROSS REFERENCE TO RELATED APPLICATIONS

This application claims priority to U.S. Provisional Patent Application Ser. No. 63/729,243, filed Dec. 6, 2024, and titled “OPTIMIZED SLC52A2 POLYNUCLEOTIDES AND EXPRESSION CASSETTES,” and U.S. Provisional Patent Application Ser. No. 63/761,068, filed Feb. 20, 2025, and titled “OPTIMIZED SLC52A2 POLYNUCLEOTIDE CONSTRUCTS AND EXPRESSION CASSETTES,” which are incorporated by reference herein in their entireties.

### INCORPORATION BY REFERENCE OF SEQUENCE LISTING

The instant application contains a Sequence Listing which has been submitted in XML and is hereby incorporated by reference in its entirety. Said XML copy, created on Dec. 5, 2025, is named “UTSD.P4462US_SEQ_LIST.xml” and is about 5500 bytes in size.

### BACKGROUND

### 1. Field

The present disclosure is directed to gene therapy compositions and methods for treating diseases associated with defective SLC52A2 gene.

### 2. Discussion of Related Art

Riboflavin transporter deficiency (RTD), formerly known as Brown-Vialetto Van Laere syndrome, is a rare recessive neurologic condition. This motor neuron disease syndrome is characterized by defective motoneurons which control speech, walking, swallowing, breathing and other general body movements. The syndrome is characterized by a phenotypic spectrum of motor, sensory, and cranial nerve neuropathy, resulting in muscle weakness, respiratory compromise, vision loss, sensorineural hearing loss, and sensory ataxia. RTD type 2 is caused by biallelic pathogenic variants in SLC52A2 gene, encoding the riboflavin transporters, hRFVT24,8. Early diagnosis is crucial, as riboflavin supplementation can significantly improve symptoms and prevent disease progression in some cases. Delayed treatment, however, may lead to irreversible neurological damage, highlighting the importance of increased awareness and early intervention. There are currently no effective cures for RTD.

### SUMMARY

In some aspects, the current disclosure encompasses a polynucleotide construct comprising a nucleic acid sequence comprising an engineered SLC52A2 gene sequence, or a cDNA thereof, or a variant thereof. In some aspects, the polynucleotide construct further comprises one or more regulatory sequences operably linked to the nucleic acid sequence. In some aspects, the one or more regulatory sequences comprise promoters, enhancers, polyadenylation signals, or terminators, or any combination thereof. In some aspects, the promoter is a UsP promoter, or a variant thereof. In some aspects, the one or more regulatory sequence comprise a UsP promoter and a polyadenylation signal. In some aspects, the nucleic acid sequence has a sequence as set forth in SEQ ID NO: 1, or a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, identical thereto.

In some aspects, the nucleic acid sequence encodes a Riboflavin Transporter 2 (RT), or any isoform thereof, or a variant thereof. In some aspects, the nucleic acid sequence encodes a polypeptide comprising an amino acid sequence as set forth in SEQ ID NO: 2, or an amino acid sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, identical thereto. In some aspects, the engineered SLC52A2 gene is codon optimized for expression in human. In some aspects, the promoter comprises a nucleic acid sequence as set forth in SEQ ID NO: 3, or a sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, identical thereto.

In some aspects, the current disclosure also encompasses a vector comprising the polynucleotide construct disclosed herein. In some aspects, the vector is a viral vector. In some aspects, the viral vector comprises a polynucleotide construct comprising a nucleic acid sequence comprising an engineered SLC52A2 gene sequence. In some aspects, the viral vector is an adeno-associated viral vector. In some aspects, the viral vector is an adeno-associated viral vector of serotype 9.

In some aspects, the current disclosure also encompasses a delivery particle comprising the polynucleotide constructs as disclosed herein, or a vector as disclosed herein. In some aspects, the current disclosure also encompasses a transgenic cell comprising the polynucleotide construct sequence or vector disclosed herein. In some aspects, the current disclosure also encompasses a pharmaceutical composition comprising a polynucleotide construct, a vector, or a transgenic cell as disclosed herein and at least one pharmaceutically acceptable excipient.

Disclosed herein is a method of treating a disease or disorder in a subject in need thereof, the method comprising administering to the subject an effective amount of the pharmaceutical compositions disclosed herein. In some aspects, the subject has a defective SLC52A2 gene. In some aspects, the subject has or is suspected of having riboflavin transporter deficiency syndrome (RTD) In some aspects, the subject is a human.

In some aspects, the current disclosure also encompasses a kit comprising the pharmaceutical composition comprising a polynucleotide construct, or a viral vector, or a transgenic cell as disclosed herein, and instructions for using the same.

### BRIEF DESCRIPTION OF THE DRAWINGS

The patent or application file contains at least one drawing executed in color. Copies of this patent or patent application publication with colored drawings will be provided by the Office upon request and payment of the necessary fee.

The following drawings form part of the present specification and are included to further demonstrate certain aspects of the present disclosure. Aspects of the present disclosure may be better understood by reference to one or more of these drawings in combination with the detailed description of specific aspects presented herein:

FIGS. 1A-1E show that SLC52A2 gene therapy rescues RTD motoneurons neurites morphology. FIG. 1A is a schematic of gene therapy construct. FIG. 1B is a representative Incucyte image of the neural network formation of CTRL and RTD motoneurons (positive for Neurolight lentivirus in red) following gene therapy. FIG. 1C are graphs depicting the quantification of neurites' length at day 45 of differentiation in treated and untreated control (**p<0.001, *p<0.01, ANOVA; mean±SEM; n=12 images, N=3 independent experiments). FIG. 1D are graphs depicting the quantification of neurites' length at day 45 of differentiation in treated and untreated RTD P1 motoneurons. FIG. 1E are graphs depicting the quantification of neurites' length at day 45 of differentiation in treated and untreated RTD P2 motoneurons (**p<0.001, *p<0.01, ANOVA; mean±SEM; n=12 images, N=3 independent experiments). Scale bar 400 um.

FIGS. 2A-2D show time-lapse imaging of the neurites' length in untreated and treated motoneurons with AAV9-SLC52A2 vector. FIG. 2A provides representative fluorescent images of Neurolight red positive CTRL neurons at different days of neuronal differentiation. FIG. 2B provides representative fluorescent images of Neurolight red positive RTD P1 at different days of neuronal differentiation, showing that at the end of in vitro neurogenesis (day 45), motoneurons of RTD patient showed longer neurites compared to those untreated. FIG. 2C provides a graph showing neurites' length of CTRL motoneurons. FIG. 2D provides a graph showing neurites' length of RTD P1 motoneurons over in vitro neurogenesis (**p=0.001, ANOVA; mean±SEM, n=12 images, N=3 independent experiments). Scale bar 400 um.

FIG. 3 shows data confirming the high quality of scAAV9/SLC52A2 vector on alkaline gel. Transduction of Lec2 cells with scAAV9/SLC52A2 vector increases SLC52A2 mRNA expression in these cells.

FIG. 4A shows histology images from both high (6×10¹¹ vg/mouse) or low (1.5×10¹¹ vg/mouse) dose of AAV9/SLC52A2 vector administered WT mice of 7 weeks. At 1-month post injection, mouse brain was harvested for RNAscope staining to detect hSLC52A2opt mRNA.

FIG. 4B shows quantitative data from the RNAscope staining. Histology images were digitized with a ScanScope slide scanner and analyzed using custom analysis settings in HALO™ Image Analysis Platform (A, scale bar 400 μm). Results are presented as % area staining positive for hSLC52A2opt mRNA by tissue region (B, mean±standard error, n=6/group). *p<0.05 compared to mice treated with vehicle.

FIGS. 5A-5E show that AAV9/SLC52A2 caused no elevation of serum toxicity panel 1-month post injection in WT (FIG. 5A: Aspartate Aminotransferase (AST) FIG. 5B: Total Bilirubin (TBIL); FIG. 5C: Blood Urea Nitrogen (BUN); FIG. 5D: Creatine Kinase (CK); FIG. 5E: Albumin (ALB). At 1-month post injection, mouse serum was collected for serum chemistry. Results are presented as mean±standard error, n=6/group. No significant difference was found between any groups.

FIGS. 5F-5J show that AAV9/SLC52A2 caused no elevation of serum toxicity panel 12-month post injection in WT (FIG. 5F: AST FIG. 5G: TBIL; FIG. 5H: BUN; FIG. 5I: CK; FIG. 5J: ALB. At 1-month post injection, mouse serum was collected for serum chemistry. Results are presented as mean±standard error, n=6/group. No significant difference was found between any groups.

FIGS. 6A-6B show that AAV9/SLC52A2 caused no effects on body weight in male (FIG. 6A) or female mice (FIG. 6B). Mice were weighed weekly for the first month following the treatment and then monthly thereafter.

The drawing figures do not limit the present disclosure to the specific aspects disclosed and described herein. The drawings are not necessarily to scale, emphasis instead being placed on clearly illustrating principles of certain aspects of the present disclosure.

### DESCRIPTION

The following detailed description references the accompanying drawings that illustrate various aspects of the present disclosure. The drawings and description are intended to describe aspects of the present disclosure in sufficient detail to enable those skilled in the art to practice the present disclosure. Other components can be utilized and changes can be made without departing from the scope of the present disclosure. The following description is, therefore, not to be taken in a limiting sense.

The current disclosure is based on the discovery that an engineered version of the SLC52A2 gene can be used with gene therapy tools to effectively alleviate defects caused by Riboflavin transporter deficiency (RTD) type 2, in motor neurons. In some aspects, disclosed herein is a method of treating RTD type 2, using gene replacement therapy, wherein a defective the SLC52A2 gene is replaced with a functional and optimized SLC52A2 gene.

The SLC52A2 gene encodes a protein that functions as a riboflavin (vitamin B2) transporter. Riboflavin is an essential nutrient involved in energy metabolism, as it is a precursor to the cofactors flavin mononucleotide (FMN) and flavin adenine dinucleotide (FAD), which are critical for various enzymatic reactions. SLC52A2 is part of the solute carrier (SLC) family, specifically the riboflavin transporter family.

Mutations in the SLC52A2 gene are associated with a rare autosomal recessive neurological disorder known as Brown-Vialetto-Van Laere syndrome 2 (BVVLS2) or riboflavin transporter deficiency 2. This disorder is characterized by progressive cranial nerve dysfunction, including hearing loss, bulbar weakness, and motor neuron disease symptoms. Early diagnosis and treatment with high-dose riboflavin supplementation have been shown to improve clinical outcomes, highlighting the importance of SLC52A2 in maintaining normal physiological function.

The protein encoded by SLC52A2 gene is localized to the cell membrane, where it acts as a high-affinity riboflavin transporter. It is crucial in maintaining systemic riboflavin homeostasis and ensuring adequate cellular availability of this essential nutrient. Research into the regulation of SLC52A2 expression and function continues to be of interest, especially in the context of metabolic disorders and potential therapeutic strategies for riboflavin deficiency-related diseases.

Riboflavin transporter deficiency (RTD), formerly known as Brown-Vialetto Van Laere syndrome, is a rare recessive neurologic condition. The disorder is a motor neuron disease characterized by defective motoneurons controlling speech, walking, swallowing, breathing and general body movements. The syndrome is characterized by a phenotypic spectrum of motor, sensory, and cranial nerve neuropathy, resulting in muscle weakness, respiratory compromise, vision loss, sensorineural hearing loss, and sensory ataxia. RTD type 2 specifically is caused by biallelic pathogenic variants in SLC52A2 gene, encoding the riboflavin transporters, hRFVT24,8.

Riboflavin (RF) is a precursor of flavin mononucleotide (FMN) and flavin adenine dinucleotide (FAD) and reduction of its intracellular availability, through defective transporters, compromises several vital processes. RF cannot be synthesized de novo and is taken from the diet through riboflavin transporters hRFVT1, 2, 3, which have different tissue distribution. Specifically, hRFVT1 is preferentially expressed in the intestinal epithelium and in placenta, hRFVT2 is localized in the central and peripheral nervous system, while hRFVT3 is mainly localized in the testis, small intestine, kidney, and placenta. Albeit empirical studies reported clinical improvement with the administration of RF, an effective cure is still lacking.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including,” and “having” are used interchangeably in this disclosure. The terms “comprising,” “including,” and “having” mean to include, but not necessarily be limited to the things so described.

The terms “or” and “and/or,” as used herein, are to be interpreted as inclusive or meaning any one, or any combination. Therefore, “A, B, or C” or “A, B, and/or C” mean any of the following: “A,” “B,” or “C”; “A and B”; “A and C”; “B and C”; “A, B, and C.” An exception to this definition will occur only when a combination of elements, functions, steps, or acts are in some way inherently mutually exclusive.

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (3rd ed. 2006); The Cambridge Dictionary of Science and Technology (Walker ed., 1990); The Glossary of Genetics, 5th Ed., R. Rieger et al. (2008), The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

The term “nucleic acid” refers to deoxyribonucleic acids (DNA), or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

A “polynucleotide construct” described herein may comprise one or more nucleic acids each encoding a polypeptide, operably linked to a promoter (i.e., in a functional relationship with) and one or more regulatory sequences. Such a polynucleotide construct may alternatively be referred to herein as a “nucleic acid construct” or “construct”.

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide construct represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide construct) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7, default value is: BLOSUM62 [b162], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lie or Val; Lys to Arg; Gln or Glu; Met to Leu or lie; Phe to Met, Leu or Tyr; Ser to Thr; Thr to Ser; Trp to Tyr; Tyr to Trp or Phe; and Val to lie or Leu.

The term “gene therapy” refers to the transfer of genetic material (e.g., DNA or RNA) of interest into a cell to treat or prevent a genetic or acquired disease or condition. The genetic material of interest encodes a product (e.g., a protein polypeptide, peptide or functional RNA) whose production in vivo is desired. For example, the genetic material of interest can encode an enzyme, hormone, receptor, or polypeptide of therapeutic value.

The term “recombinant” as used herein to describe a nucleic acid molecule, means a polynucleotide construct of genomic, cDNA, viral, semisynthetic, and/or synthetic origin, which, by virtue of its origin or manipulation, is not associated with all or a portion of the polynucleotide construct with which it is associated in nature.

As used herein, “treatment,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition, and/or the remission of the disease, disorder or condition.

As used herein, “prevent,” or “prevention” refers to eliminating or delaying the onset of a particular disease, disorder or physiological condition, or to the reduction of the degree of severity of a particular disease, disorder or physiological condition, relative to the time and/or degree of onset or severity in the absence of intervention.

The term “effective amount,” or “therapeutically effective amount” refers to an amount sufficient to effect beneficial or desirable biological and/or clinical results. The term “therapeutically effective amount,” as used herein, means an amount of a compound or combination of compounds that ameliorates, attenuates, or eliminates one or more symptoms of cancer or prevents or delays the onset of one or more symptoms of cancer as defined herein.

As used herein, “individual,” “subject,” “host,” and “patient” can be used interchangeably herein and refer to any mammalian subject for whom diagnosis, treatment, prophylaxis or therapy is desired, for example, humans, pets, livestock, horses or other animals. As used herein, the term “subject,” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some aspects, the subject can be a human. In other aspects, the subject can be a human in need of treatment for RTD syndrome.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

### II. Compositions

In some aspects, provided herein are polynucleotide constructs comprising a nucleic acid sequence comprising an engineered SLC52A2 gene (NCBI Gene ID human: 79581; NCBI Gene ID mouse: 52710), or a cDNA thereof, or a variant thereof. In some aspects, the SLC52A2 gene, or a cDNA thereof, or a variant thereof, is codon optimized to be expressed in a suitable host, for example, a human.

In some aspects, the polynucleotide construct comprises a nucleic acid sequence as set forth in SEQ ID NO: 1, or a sequence with at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, (or any range derivable therein) identity thereto.

SEQ ID NO: 1
ATGGCTGCCCCAACTCCCGCAAGACCGGTGTTGACCCATCTCCTCGTGGCCCTGT
TCGGGATGGGATCATGGGCGGCCGTGAATGGCATTTGGGTGGAGCTGCCCGTGGTGGTC
AAGGAGCTTCCCGAGGGATGGTCCCTCCCGTCCTACGTGTCCGTCCTGGTGGCCCTGGG
AAACCTGGGCCTCCTGGTGGTCACCCTGTGGAGAAGGCTCGCCCCTGGAAAGGATGAACA
GGTCCCGATTCGCGTGGTCCAGGTCCTGGGGATGGTCGGCACCGCTCTGCTGGCCTCCC
TGTGGCACCACGTGGCACCAGTCGCGGGACAGCTGCACTCCGTGGCGTTCTTAGCCCTTG
CCTTCGTGCTCGCCTTGGCCTGCTGTGCCTCGAACGTGACGTTCCTGCCCTTCCTGTCCC
ATCTTCCCCCCCGGTTTCTGCGGTCGTTCTTCCTGGGACAGGGACTGAGCGCCCTGCTGC
CTTGTGTGCTGGCCCTTGTGCAAGGAGTGGGACGCCTGGAATGTCCGCCGGCACCCATCA
ACGGGACTCCAGGCCCTCCTCTGGACTTCCTGGAACGGTTCCCGGCCTCCACCTTTTTCT
GGGCGCTGACAGCGTTGCTTGTGGCGTCAGCTGCAGCGTTTCAGGGCCTTCTGCTCCTCC
TCCCTCCTCCGCCTTCCGTGCCGACTGGGGAACTGGGCTCTGGGCTCCAAGTCGGAGCC
CCAGGAGCTGAGGAAGAAGTGGAGGAATCCTCGCCGTTGCAAGAGCCCCCGTCACAAGC
GGCTGGCACCACTCCCGGTCCTGACCCCAAAGCATACCAGCTGCTGTCGGCTAGGAGCG
CCTGTCTGCTGGGACTCCTCGCTGCGACTAACGCGCTGACCAACGGAGTGCTCCCAGCC
GTGCAGTCCTTCTCCTGCCTGCCGTACGGTCGCCTGGCCTACCACCTGGCCGTGGTGCTG
GGTAGCGCCGCTAACCCTCTGGCCTGCTTCCTGGCCATGGGCGTGTTGTGCCGGTCACTG
GCTGGGCTGGGAGGACTTAGCCTCCTGGGAGTGTTCTGCGGTGGCTACCTGATGGCCCT
GGCCGTCTTGTCCCCCTGCCCTCCTCTCGTGGGAACCTCCGCCGGAGTGGTCTTGGTGGT
GCTGTCGTGGGTGCTGTGTCTCGGGGTGTTCAGCTACGTGAAGGTTGCCGCGTCAAGCCT
GCTCCACGGTGGCGGAAGGCCTGCACTGCTGGCAGCAGGCGTAGCCATCCAAGTCGGCA
GCCTACTCGGAGCCGTGGCCATGTTCCCGCCGACCTCCATCTATCACGTCTTTCATTCCCG
GAAGGACTGCGCCGATCCATGCGACAGCTGA

In some aspects, the nucleic acid sequence disclosed herein encodes a riboflavin transporter, or an isoform thereof, or a functional variant thereof. In some aspects, the nucleic acid sequence disclosed herein encodes a polypeptide comprising an amino acid sequence as set forth in SEQ ID NO: 2, or an amino acid sequence at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, (or any range derivable therein) identical thereto.

SEQ ID NO: 2
MAAPTPARPVLTHLLVALFGMGSWAAVNGIWVELPVVVKELPEGWSLPSY
VSVLVALGNLGLLVVTLWRRLAPGKDEQVPIRVVQVLGMVGTALLASLWH
HVAPVAGQLHSVAFLALAFVLALACCASNVTFLPFLSHLPPRFLRSFFLG
QGLSALLPCVLALVQGVGRLECPPAPINGTPGPPLDFLERFPASTFFWAL
TALLVASAAAFQGLLLLLPPPPSVPTGELGSGLQVGAPGAEEEVEESSPL
QEPPSQAAGTTPGPDPKAYQLLSARSACLLGLLAATNALTNGVLPAVQSF
SCLPYGRLAYHLAVVLGSAANPLACFLAMGVLCRSLAGLGGLSLLGVFCG
GYLMALAVLSPCPPLVGTSAGVVLVVLSWVLCLGVFSYVKVAASSLLHGG
GRPALLAAGVAIQVGSLLGAVAMFPPTSIYHVFHSRKDCADPCDS

In some aspects, the nucleic acid sequences disclosed herein may be codon optimized. Codon optimization is a process used to modify a nucleic acid sequence to enhance its expression in a host cell while maintaining the native amino acid sequence. This process involves replacing codons of the native sequence with those most frequently used in the genes of the host organism. The optimization can be applied to specific regions, including upstream (5′) or downstream (3′) regions of an open reading frame (ORF), ensuring proper folding and efficient translation. Codon optimization methods are well-established in the field and aim to achieve multiple goals, such as matching codon frequencies between target and host organisms, biasing nucleotide content to alter stability or reduce secondary structures, and minimizing tandem repeat codons or base runs that may impair gene construction or expression. Other objectives include modifying transcriptional and translational control regions, inserting or removing protein signaling sequences, and adjusting translational rates to ensure proper protein folding.

Codon optimization can also serve functional purposes, such as eliminating cryptic start codons, splice sites, or other destabilizing elements that could interfere with transcription or translation. Additionally, optimization may reduce the frequency of CpG dinucleotides, which are known to provoke heightened immune responses, particularly in therapeutic or vaccine applications. Another benefit of codon optimization is its ability to distinguish a transgene from endogenous sequences, facilitating molecular tracking of transgene DNA and/or expressed mRNA. Codon usage may be assessed using the Codon Adaptation Index (CAI), which measures the deviation of a coding sequence from a reference gene set, and codon usage tables, which are available through databases such as the Codon Usage Database. Tools and services for codon optimization, including ATUM (Menlo Park, CA, USA), GeneArt (Life Technologies), DNA2.0, OptimumGene (GenScript), and algorithms such as DNA Works v3.2.3, provide various approaches for optimizing expression in specific species.

In addition to codon optimization additional changes are envisaged that may remove cryptic donor splice sites, resulting in more predictable and higher expression in vivo. Additionally, the sequence may be designed to have a more evenly distributed GC content, which leads to a more uniform melting temperature and improved transcription/translation efficiency.

Following optimization, the modified polynucleotide components may be reconstituted and integrated into vectors, such as plasmids, or viruses, viral vectors, cosmids, and artificial chromosomes, for further applications. These optimized sequences can enhance gene therapy vectors, improving their stability, translational efficiency, and overall efficacy in therapeutic and research settings.

In some aspects, the polynucleotide construct disclosed herein further comprises one or more regulatory sequence operably linked to the nucleic acid sequence. As used herein, “regulatory elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary, or preferred for regulating or allowing expression of the gene or transcribable DNA sequence in a cell. Such additional regulatory element(s) can be optional and used to enhance or optimize expression of the gene or transcribable DNA sequence. A regulatory sequence can, for example, be inducible, non-inducible, constitutive, cell-cycle regulated, metabolically regulated, and the like. A regulatory sequence may be a promoter. As used herein, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. An “individual” or “subject,” as used interchangeably herein, is a mammal. In certain aspects, the individual or subject is a human.

In some aspects, a promoter is an inducible promoter, a constitutive promoter, a mammalian cell promoter, a viral promoter, a chimeric promoter, an engineered promoter, a tissue-specific promoter, or any other type of promoter known in the art. In some aspects, a promoter is a RNA polymerase II promoter, such as a mammalian RNA polymerase II promoter. In some aspects, a promoter is a RNA polymerase Ill promoter, including, but not limited to, a HI promoter, a human U6 promoter, a mouse U6 promoter, or a swine U6 promoter. A promoter will generally be one that is able to promote transcription in a mammalian cell. A variety of promoters are known in the art, which in some aspects, can be used herein. Non-limiting examples of promoters that can be used herein in some aspects include: human EF1a, human cytomegalovirus (CMV), human ubiquitin C (UBC), mouse phosphoglycerate kinase 1, polyoma adenovirus, simian virus 40 (SV40), β-globin, β-actin, α-fetoprotein, γ-globin, μ-interferon, γ-glutamyl transferase, mouse mammary tumor virus (MMTV), Rous sarcoma virus, rat insulin, glyceraldehyde-3-phosphate dehydrogenase, metallothionein II (MT 1II), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class II, MHC class II HLA-DRa, muscle creatine kinase, prealbumin (transthyretin), elastase 1, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GAL V) promoters. In some aspects, a promoter is the CMV immediate early promoter. In some aspects, the promoter is a CAG promoter and/or a CAG/CBA promoter.

The term “constitutive” promoter refers to a nucleotide sequence that, when operably linked with a nucleic acid encoding a gene, causes RNA to be transcribed from the nucleic acid in a cell under most or all physiological conditions. Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated herein by reference for the purposes described herein), the SV 40 promoter, the dihydrofolate reductase promoter, the beta-actin promoter, the phosphoglycerol kinase (PGK) promoter, and the EFI-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art. Examples of inducible promoters regulated by exogenously supplied compounds include the zinc-inducible sheep metallothionein (MT) promoter, the dexamethasone (Dex) inducible mouse mammary tumor virus (MMTV) promoter, the T7 polymerase promoter system; the ecdysone insect promoter, the tetracycline-repressible system, the tetracycline-inducible system, the RU486-inducible system, and the rapamycin-inducible system.

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter). In some aspects, regulatory and/or control sequences impart tissue-specific gene expression capabilities. In some cases, tissue-specific regulatory and/or control sequences bind tissue-specific transcription factors that induce transcription in a tissue-specific manner. In some aspects, a tissue-specific promoter is a neuron-specific promoter. In some aspects, a tissue-specific promoter is hematopoietic lineage cell-specific promoter. In some aspects, a tissue-specific promoter is an immune cell-specific promoter.

In some aspects, the promoter is a weak promoter, a moderately expressing promoter, or a strong constitutive or inducible promoter. In some aspects, the promoter is a moderately expressing promoter. In some aspects, the promoter is a JeT promoter, or a variant thereof. In some aspects, the promoter is a Ubiquitous Synthetic Promoter (USP), or a variant thereof, which is commonly used in Adeno-Associated Virus (AAV) vectors to drive strong, constitutive gene expression across various tissues. The UsP promoter is derived from the JeT promoter and includes a synthetic intron to enhance expression levels. This promoter is particularly useful in self-complementary AAV (scAAV) vectors due to its compact size and ability to drive ubiquitous gene expression (“Novel Vector Design and Hexosaminidase Variant Enabling Self-Complementary AAV9-Mediated Gene Therapy for Tay-Sachs and Sandhoff Diseases,” published in Human Gene Therapy in 2017). In some aspects, the USP promoter comprises a nucleic acid sequence as set forth in SEQ ID NO: 3, or a sequence with at least about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, (or any range derivable therein) identity thereto.

SEQ ID NO: 3
GGGCGGAGTTAGGGCGGAGCCAATCAGCGTGCGCCGTTCCGAAAGTTGCC
TTTTATGGCTGGGCGGAGAATGGGCGGTGAACGCCGATGATTATATAAGG
ACGCGCCGGGTGTGGCACAGCTAGTTCCGTCGCAGCCGGGATTTGGGTCG
CGGTTCTTGTTTGTCTGTGATCGTCACTTGGTAAGTCACTGACTGTCTAT
GCCTGGGAAAGGGGGGCAGGAGATGGGGCAGTGCAGGAAAAGTGGCACTA
TGAACCCTGCAGCCCTAGGAATGCATCTAGACAATTGTACTAACCTTCTT
CTCTTTCCTCTCCTGACAG

In some aspects, additional regulatory sequences may be operably linked to the polynucleotide construct disclosed here. In some aspects, the additional regulatory sequence may comprise one or more regulatory elements described herein in the context of AAV constructs.

In some aspects, the polynucleotide construct disclosed herein may be comprised within a vector for delivery, and/or may be delivered in a delivery particle. Vectors comprising polynucleotide constructs according to the present disclosure include all those known in the art, including cosmids, plasmids (e.g., naked or contained in liposomes), and viral constructs (e.g., lentiviral, retroviral, adenoviral, and adeno associated viral constructs) that can incorporate a polynucleotide construct encoding a polypeptide described herein, or a variant thereof. Those of skill in the art will be capable of selecting suitable constructs, as well as cells, for making any of the polynucleotide constructs described herein. In some aspects, the construct is a plasmid (i.e., a circular DNA molecule that can autonomously replicate inside a cell). In some aspects, the construct can be a cosmid (e.g., pWE or sCos series).

In some aspects, a vector is a viral vector. In some aspects, a viral construct is a lentivirus, retrovirus, adenovirus, or adeno-associated virus construct. In some aspects, a construct is an adeno-associated virus (AAV) construct. In some aspects, a viral construct is an adenovirus construct. In some aspects, a viral construct may also be based on or derived from an alphavirus. Pseudotyped viruses may be formed by combining alphaviral envelope glycoproteins and retroviral capsids.

In some aspects, constructs provided herein can be of different sizes. In some aspects, a construct is a plasmid and can include a total length of up to about 1 kb, up to about 2 kb, up to about 3 kb, up to about 4 kb, up to about 5 kb, up to about 6 kb, up to about 7 kb, up to about 8 kb, up to about 9 kb, up to about 10 kb, up to about 11 kb, up to about 12 kb, up to about 13 kb, up to about 14 kb, or up to about 15 kb. In some aspects, a construct is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some aspects, a construct is a viral construct and can have a total number of nucleotides of up to 10 kb. In some aspects, a viral construct can have a total number of nucleotides in the range of about 4.5 kb to 5 kb, or about 4.7 kb. In some aspects, a viral construct can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 1 O kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some aspects, a construct is a lentivirus construct and can have a total number of nucleotides of up to 8 kb. In some examples, a lentivirus construct can have a total number of nucleotides of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 8 kb, about 6 kb to about 7 kb, or about 7 kb to about 8 kb.

In some aspects, a construct is an adenovirus construct and can have a total number of nucleotides of up to 8 kb. In some aspects, an adenovirus construct can have a total number of nucleotides in the range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 3 kb to about 4 kb, about 3 kb to about 5 kb, about 3 kb to about 6 kb, about 3 kb to about 7 kb, about 3 kb to about 8 kb, about 4 kb to about 5 kb, about 4 kb to about 6 kb, about 4 kb to about 7 kb, about 4 kb to about 8 kb, about 5 kb to about 6 kb, about 5 kb to about 7 kb, about 5 kb to about 8 kb, about 6 kb to about 7 kb, about 6 kb to about 8 kb, or about 7 kb to about 8 kb.

Any of the constructs described herein can further include a control sequence, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or additional untranslated regions which may house pre- or post-transcriptional regulatory and/or control elements. In some aspects, a promoter can be a native promoter, a constitutive promoter, an inducible promoter, and/or a tissue-specific promoter. Non-limiting examples of control sequences are described herein.

### AAV Particles

In some aspects, AAV particles can be described as having a serotype, which is a description of the construct strain and the capsid strain. For example, in some aspects an AAV particle may be described as AAV2, wherein the particle has an AAV2 capsid and a construct that comprises characteristic AAV2 Inverted Terminal Repeats (ITRs). In some aspects, an AAV particle may be described as a pseudotype, wherein the capsid and construct are derived from different AAV strains, for example, AAV2/9 would refer to an AAV particle that comprises a construct utilizing the AAV2 ITRs and an AAV9 capsid. Additional examples of pseudotyped AAV vectors include, but are not limited to, AAV2/1, AAV2/2, AAV2/3, AAV2/4, AAV2/5, AAV2/6, AAV2/7, AAV2/8, and AAV2/9.

In some aspects, AAV particles suitable for use according to the present disclosure may comprise or be derived from any natural or recombinant AAV serotype. In some aspects, an AAV according to the present disclosure can be selected from natural serotypes such as AAV1, AAV2, AAV3, AAV4, AAV5, AAV6, AAV7, AAV8, AAV9, AAV10, AAV11, and AAV12; or pseudotypes, chimeras, and variants thereof.

As used herein, the term “chimera” when referring to an AAV vector, or a “chimeric AAV vector”, refers to an AAV vector which comprises a capsid containing VP1, VP2 and VP3 proteins from at least two different AAV serotypes; or alternatively, which comprises VP1, VP2 and VP3 proteins, at least one of which comprises at least a portion from another AAV serotype. Examples of chimeric AAV vectors include, but are not limited to, AAV-DJ, AAV-DJ/8, AAV2G9, AAV2i8, AAV2i8G9, AAV8G9, and AAV9i1.

In some aspects, an AAV serotype and/or pseudotype according to the present invention is selected from the group comprising or consisting of AAV1, AAV2, AAV3, AAV 4, AAV5, AAV6, AAV7, AAV8, AAV9, AAV10, AAV11, AAV12, AAV106.1/hu.37, AAV114.3/hu.40, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2i8, AAV2i8G9, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV2.5T, AAV27.3, AAV29.3/bb.1, AAV29.5/bb.2, AAV2G9, AAV3B, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/rh.64, AAV4-9/rh.54, AAV52.1/hu.20,AAV52/hu.19, AAV5-22/rh.58, AAV5-3/rh.57, AAV54.1/hu.21, AAV54.2/hu.22,AAV54.4R/hu.27, AAV54.5/hu.23, AAV54.7/hu.24, AAV58.2/hu.25, AAV6.1, AAV6.1.2, AAV6.2, AAV7m8, AAV7.2, AAV7.3/hu.7, AAV-8b, AAV8G9, AAV-8h, AAV9i1, AAV9.11, AAV9.13, AAV9.16, AAV9.24, AAV9.45, AAV9.47, AAV9.61, AAV9.68, AAV9.84, AAV9.9, AAVcy.2, AAVcy.3, AAVcy.4, AAVcy.5, AAVcy.5R1, AAVcy.5R2, AAVcy.5R3, AAVcy.5R4, AAVcy.6, AAVhu.1, AAVhu.2, AAVhu.3, AAVhu.4, AAVhu.5, AAVhu.6, AAVhu.7, AAVhu.8, AAVhu.9, AAVhu.10, AAVhu.11, AAVhu.12, AAVhu.13, AAVhu.14/9, AAVhu.15, AAVhu.16, AAVhu.17, AAVhu.18, AAVhu.19, AAVhu.20, AAVhu.21, AAVhu.22, AAVhu.23.2, AAVhu.24, AAVhu.25, AVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.53, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVpi.1, AAVpi.2, AAVpi.3, AAVrh.2, AAVrh.2R, AAVrh.8, AAVrh.8R, AAVrh8R R533A mutant, AAVrh8R A586R mutant, AAVrh.10, AAVrh.12, AAVrh.13, AAVrh. 13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAV-PHP.B, AAVPHP.A, AAV-G2B-26, AAV-G2B-13, AAV-TH1.1-32, AAVTH1.1-35, AAV-PHP.B2, AAV-PHP.B3, AAV-PHP.N/PHP.B-DGT, AAV-PHP.B-EST, AAV-PHP.B-GGT, AAV-PHP.BATP, AAV-PHP.B-ATT-T, AAV-PHP.B-DGT-T, AAV-PHP.B-GGT-T, AAV-PHP.B-SGS, AAV-PHP.B-AQP, AAV-PHP.B-QQP, AAV-PHP.B-SNP(3), AAV-PHP.B-SNP, AAV-PHP.B-QGT, AAV-PHP.B-NQT, AAV-PHP.B-EGS, AAV-PHP.BSGN, AAV-PHP.B-EGT, AAV-PHP.B-DST, AAV-PHP.BDST, AAV-PHP.B-STP, AAV-PHP.B-PQP, AAV-PHP.BSQP, AAV-PHP.B-Q1P, AAV-PHP.B-TMP, AAV-PHP.BTTP, AAV-PHP.S/G2A12, AAV-G2A15/G2A3, AAV-G2B4, AAV-G2B5, PHP.S, AAAV, AAV A3.3, AAV A3.4, AAV A3.5, AAV A3.7, AAV CBr-7.3, AAV CBr-7.1, AAV CBr-7.10, AAV CBr-7.2, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-N4, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKdB4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-M9, AAV CLv-R6, AAV CLv-1, AAV CLv1-1, AAV CLv1-10, AAV CLv1-2, AAV CLv-12, AAV CLv1-3, AAV CLv-13, AAV CLv1-4, AAV CLv1-7, AAV CLv1-8, AAV CLv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLvM7, AAV CLv-M8, AAV CLv-R1, AAV CLv-R2, AAV CLv-R3, AAV CLv-R4, AAV CLv-R5, AAV CLv-R7, AAV CLv-R8, AAV CLv-R9, AAV CSp-8.10, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.2, AAV CSp-8.4, AAV CSp-8.5, AAV CSp-8.6, AAV CSp-8.7, AAV CSp-8.8, AAV CSp-8.9, AAV CSp-9, AAVLK08, AAV-LK15, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV.VR-355, AAV-b, AAVC1, AAVC2, AAVC5, AAVCh.5, AAVCh.5R1, AAV-DJ, AAV-DJ8, AAVF1/HSC1, AAVF11/HSC11, AAVF12/HSC12, AAVF13/HSC13, AAVF14/HSC14, AVF15/HSC15, AAVF16/HSC16, AAVF17/HSC17, AAVF2/HSC2, AAVF3, AAVF3/HSC3, AAVF4/HSC4, AAVF5, AAVF5/HSC5, AAVF6/HSC6, AAVF7/HSC7, AAVF8/HSC8, AAVF9/HSC9, AAV-h, AAVH-1/hu.1,AAVH2,AAVH-5/hu.3,AAVH6,AAVhE1.1, AAVhEr1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVLG-10/rh.40, AAVLG-4/rh.38, AAVLG-9/hu.39, AAVLG-9/hu.39, AAV-LK01, AAV-LKO2, AAV-LKO3, AAV-LKO3, AAV-LKO4, AAV-LK05, AAV-LK06, AAVLK07, AAV-LK09, AAV-LK10, AAV-LK11, AAV-LK12, AAV-LK13, AAV-LK14, AAV-LK16, AAV-LK17, AAVLK18, AAV-LK19, AAVN721-8/rh.43, AAV-PAEC, AAVPAEC12, AAV-PAEC11, AAV-PAEC2, AAV-PAEC4, AAVPAEC6, AAV-PAEC7, AAV-PAECS, Anc80, Anc80L65, Anc81, Anc82, Anc83, Anc84, Anc94, Anc110, Anc113, Anc126, Anc127, BAAV, BNP61 AAV, BNP62 AAV, BNP63 AAV, bovine AAV, caprine AAV, Japanese AAV10 serotype, UPENN AAV10, VOY101, and VOY201.

In some aspects, an AAV is an AAV variant that has been genetically modified, e.g., by substitution, deletion or addition of one or several amino acid residues in one or more capsid proteins. Examples of such variants include, but are not limited to, AAV2 with one or more of Y444F, Y500F, Y730F and/or S662V mutations; AAV3 with one or more of Y705F, Y731F and/or T492V mutations; and AAV6 with one or more of S663V and/or T492V mutations.

In some aspects, an AAV capsid is modified to comprise at least one surface-bound saccharide or a variant thereof. As used herein, the term “surface-bound”, when referring to the at least one saccharide, means that said at least one saccharide is bound to and exposed at the outer surface of the AAV vector. Suitable examples of saccharides include, but are not limited to, monosaccharides, oligosaccharides, polysaccharides, and variants thereof.

### AAV Constructs

In some aspects, a polynucleotide construct comprises one or more components derived from or modified from a naturally occurring AAV genomic construct. In some aspects, a sequence derived from an AAV construct is an AAV1 construct, an AAV2 construct, an AAV3 construct, an AAV4 construct, an AAV5 construct, an AAV6 construct, an AAV7 construct, an AAV8 construct, an AAV DJ/8 construct, an AAV9 construct, an AAV2.7m8 construct, an AAV8BP2 construct, an AAV293 construct, an AAVPhp.B construct, orAAVPhp.eB construct (see e.g., Chan et al., 2017). Additional exemplary AAV constructs that can be used herein are known in the art.

In some aspects, AAV derived sequences (e.g., which are comprised in a polynucleotide construct) typically include the cis-acting 5′ and 3′ ITR sequences. Typical AAV2-derived ITR sequences are about 145 nucleotides in length. In some aspects, at least or exactly 80% of a typical ITR sequence (e.g., at least or exactly 85%, at least or exactly 90%, at least or exactly 95%, or at least or exactly 100%, etc.) is incorporated into a construct provided herein. The ability to modify these ITR sequences is within the skill of the art. In some aspects, any of the coding sequences and/or constructs described herein are flanked by 5′ and 3′ AAV ITR sequences. The AAV ITR sequences may be obtained from any known AAV, including presently identified AAV types.

In some aspects, polynucleotide constructs described in accordance with this disclosure and in a pattern known to the art are typically comprised of, a coding sequence or a portion thereof, at least one and/or control sequence, and optionally 5′ and 3′ AAV inverted terminal repeats (ITRs). In some aspects, provided constructs can be packaged into a capsid to create an AAV particle. An AAV particle may be delivered to a selected target cell. In some aspects, provided constructs comprise an additional optional coding sequence that is a nucleic acid sequence (e.g., inhibitory nucleic acid sequence), heterologous to the construct sequences, which encodes a polypeptide, protein, functional RNA molecule (e.g., miRNA, miRNA inhibitor) or other gene product, of interest. In some aspects, a nucleic acid coding sequence is operatively linked to and/or control components in a manner that permits coding sequence transcription, translation, and/or expression in a cell of a target tissue.

In some aspects, an unmodified AAV endogenous genome includes two open reading frames, “cap” and “rep,” which are flanked by ITRs. In some aspects, recombinant AAV constructs similarly comprise one or more open reading frames flanked by ITR sequences. In some aspects, an AAV construct also comprises conventional control elements that are operably linked to the coding sequence in a manner that permits its transcription, translation and/or expression in a cell transfected with the polynucleotide construct or infected with a virus particle produced by the disclosure. In some aspects, an AAV construct optionally comprises a promoter, an enhancer, an untranslated region (e.g., a 5′ UTR, 3′ UTR), a Kozak sequence, an internal ribosomal entry site (IRES), splicing sites (e.g., an acceptor site, a donor site), a polyadenylation site, or any combination thereof.

In some aspects, a construct is an AAV construct. In some aspects, an AAV construct can include at least 500 bp, at least 1 kb, at least 1.5 kb, at least 2 kb, at least 2.5 kb, at least 3 kb, at least 3.5 kb, at least 4 kb, at least 4.5 kb, or at least 4.7 kb. In some aspects, an AAV construct can include at most 7.5 kb, at most 7 kb, at most 6.5 kb, at most 6 kb, at most 5.5 kb, at most 5 kb, at most 4.5 kb, at most 4 kb, at most 3.5 kb, at most 3 kb, or at most 2.5 kb. In some aspects, an AAV construct can include about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5 kb, about 3 kb to about 4 kb, about 3 kb to about 5 kb, or about 4 kb to about 5 kb.

Any of the constructs described herein can further include regulatory and/or control sequences, e.g., a control sequence selected from the group of a transcription initiation sequence, a transcription termination sequence, a promoter sequence, an enhancer sequence, an RNA splicing sequence, a polyadenylation (poly(A)) sequence, a Kozak consensus sequence, and/or any combination thereof. In some aspects, a promoter can be a native promoter, a constitutive promoter, an inducible promoter, and/or a tissue-specific promoter. Non-limiting examples of control sequences are described herein and others are known in the art

### AAV Capsids

In some aspects, the present disclosure provides one or more polynucleotide constructs packaged into an AAV capsid. In some aspects, an AAV capsid is from or is derived from an AAV capsid of an AAV2, 3, 4, 5, 6, 7, 8, 9, 10, rh8, rhl0, rh39, rh43 or Ancestral serotype, or one or more hybrids thereof. In some aspects, an AAV capsid is from an AAV ancestral serotype. In some aspects, an AAV capsid is an ancestral (Anc) AAV capsid. An Anc capsid is created from a construct sequence that is constructed using evolutionary probabilities and evolutionary modeling to determine a probable ancestral sequence. Thus, an Anc capsid/construct sequence is not known to have existed in nature. As provided herein, in some aspects, any combination of AAV capsids and AAV constructs (e.g., comprising AAV ITRs) may be used in recombinant AAV particles of the present disclosure.

### Exemplary AAV Construct Components

### Inverted Terminal Repeat Sequences (ITRs)

AAV derived sequences of a construct typically comprises the cis-acting 5′ and 3′ ITRs. Generally, ITRs are able to form a hairpin. The ability to form a hairpin can contribute to an ITRs ability to self-prime, allowing primase-independent synthesis of a second DNA strand. ITRs can also aid in efficient encapsidation of an AAV construct in an AAV particle.

An AAV particle of the present disclosure can comprise an AAV construct comprising a coding sequence and associated elements flanked by a 5 and a 3′ AAV ITR sequences. In some aspects, an ITR is or comprises about 130 nucleic acids. In some aspects, an ITR is or comprises about 145 nucleic acids. In some aspects, all or substantially all of a sequence encoding an ITR is used. In some aspects, an AAV ITR sequence may be obtained from any known AAV, including presently identified mammalian AAV types. In some aspects an ITR is an AAV2 ITR. In some aspects, an ITR is an AAV9 ITR.

A non-limiting example of a polynucleotide construct of the present disclosure is a “cisacting” construct comprising a coding sequence, in which said sequence and any associated regulatory elements are flanked by 5′ or “left” and 3′ or “right” AAV ITR sequences. 5′ and left designations refer to a position of an ITR sequence relative to an entire construct, read left to right, in a sense direction. For example, in some aspects, a 5′ or left ITR is an ITR that is closest to a promoter (e.g., as opposed to a polyadenylation sequence) for a given construct, when a construct is depicted in a sense orientation, linearly. Concurrently, 3′ and right designations refer to a position of an ITR sequence relative to an entire construct, read left to right, in a sense direction. For example, in some aspects, a 3′ or right ITR is an ITR that is closest to a polyadenylation sequence and/or stop codon (e.g., as opposed to a promoter sequence) for a given construct, when a construct is depicted in a sense orientation, linearly. In general, ITRs as provided herein are depicted in 5′ to 3′ order in accordance with a sense strand. Accordingly, one of skill in the art will appreciate that a 5′ or “left” orientation ITR can also be depicted as a 3′ or “right” ITR when converting from sense to anti sense direction. Further, it is well within the ability of one of skill in the art to transform a given sense ITR sequence (e.g., a 5/left AAV ITR) into an antisense sequence (e.g., 3/right ITR sequence). One of ordinary skill in the art would understand how to modify a given ITR sequence for use as either a 5/left or 3/right ITR, or an antisense version thereof.

### Promoters

In some aspects, a construct (e.g., an AAV construct) comprises a promoter. The term “promoter” refers to a DNA sequence recognized by enzymes/proteins that can promote and/or initiate transcription of an operably linked gene. For example, a promoter typically refers to, e.g., a nucleotide sequence to which an RNA polymerase and/or any associated factor binds and from which it can initiate transcription. Thus, in some aspects, a construct (e.g., an AAV construct) comprises a promoter operably linked to one of the non-limiting example promoters described herein.

In some aspects, a promoter is an inducible promoter, a constitutive promoter, a mammalian cell promoter, a viral promoter, a chimeric promoter, an engineered promoter, a tissue-specific promoter, or any other type of promoter known in the art. In some aspects, a promoter is a RNA polymerase II promoter, such as a mammalian RNA polymerase II promoter. In some aspects, a promoter is a RNA polymerase Ill promoter, including, but not limited to, a HI promoter, a human U6 promoter, a mouse U6 promoter, or a swine U6 promoter. A promoter will generally be one that is able to promote transcription in a mammalian cell.

A variety of promoters are known in the art, which in some aspects, can be used herein. Nonlimiting examples of promoters that can be used herein in some aspects include: human EF1a, human cytomegalovirus (CMV) (U.S. Pat. No. 5,168,062, which is incorporated herein by reference for the purposes described herein), human ubiquitin C (UBC), mouse phosphoglycerate kinase 1, polyoma adenovirus, simian virus 40 (SV40), β-globin, β-actin, α-fetoprotein, γ-globin, μ-interferon, γ-glutamyl transferase, mouse mammary tumor virus (MMTV), Rous sarcoma virus, rat insulin, glyceraldehyde-3-phosphate dehydrogenase, metallothionein II (MT 1l), amylase, cathepsin, MI muscarinic receptor, retroviral LTR (e.g., human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2K b, HSP70, proliferin, tumor necrosis factor, thyroid stimulating hormone a gene, immunoglobulin light chain, T-cell receptor, HLA DQa and DQ, interleukin-2 receptor, MHC class 1l, MHC class II HLA-DRa, muscle creatine kinase, prealbumin (transthyretin), elastase 1, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GAL V) promoters. Additional examples of promoters are known in the art. See, e.g., Lodish, Molecular Cell Biology, Freeman and Company, New York 2007, each of which is incorporated herein by reference for the purposes described herein. In some aspects, a promoter is the CMV immediate early promoter. In some aspects, the promoter is a CAG promoter and/or a CAG/CBA promoter.

The term “constitutive” promoter refers to a nucleotide sequence that, when operably linked with a nucleic acid encoding a gene, causes RNA to be transcribed from the nucleic acid in a cell under most or all physiological conditions. Examples of constitutive promoters include, without limitation, the retroviral Rous sarcoma virus (RSV) LTR promoter, the cytomegalovirus (CMV) promoter (see, e.g., Boshart et al., Cell 41:521-530, 1985, which is incorporated herein by reference for the purposes described herein), the SV 40 promoter, the dihydrofolate reductase promoter, the beta-actin promoter, the phosphoglycerol kinase (PGK) promoter, and the EFI-alpha promoter (Invitrogen).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art. Examples of inducible promoters regulated by exogenously supplied compounds include the zinc-inducible sheep metallothionein (MT) promoter, the dexamethasone (Dex) inducible mouse mammary tumor virus (MMTV) promoter, the T7 polymerase promoter system (see e.g., WO 98/10088, which is incorporated herein by reference for the purposes described herein); the ecdysone insect promoter (see e.g., No et al., Proc. Natl. Acad Sci. U.S.A 93:3346-3351, 1996, which is incorporated herein by reference for the purposes described herein), the tetracycline-repressible system (see e.g., Gossen et al., Proc. Natl. Acad Sci. U.S.A 89:5547-5551, 1992, which is incorporated herein by reference for the purposes described herein), the tetracycline-inducible system (see e.g., Gossen et al., Science 268: 1766-1769, 1995, see also Harvey et al., Curr. Opin. Chem. Biol. 2:512-518, 1998, each of which is incorporated herein by reference for the purposes described herein), the RU486-inducible system (see e.g., Wang et al., Nat. Biotech. 15:239-243, 1997, and Wang et al., Gene Ther. 4:432-441, 1997, each of which is incorporated herein by reference for the purposes described herein), and the rapamycin-inducible system (see e.g., Magari et al., J Clin. Invest. 100:2865-2872, 1997, which is incorporated herein by reference for the purposes described herein).

The term “tissue-specific” promoter refers to a promoter that is active only in certain specific cell types and/or tissues (e.g., transcription of a specific gene occurs only within cells expressing transcription regulatory and/or control proteins that bind to the tissue-specific promoter). In some aspects, regulatory and/or control sequences impart tissue-specific gene expression capabilities. In some cases, tissue-specific regulatory and/or control sequences bind tissue-specific transcription factors that induce transcription in a tissue-specific manner. In some aspects, a tissue-specific promoter is a neuron-specific promoter. In some aspects, a tissue-specific promoter is hematopoietic lineage cell-specific promoter. In some aspects, a tissue-specific promoter is an immune cell-specific promoter.

In some aspects, the promoter is a UsP promoter.

### Enhancers

In some aspects, a construct can include an enhancer sequence. The term “enhancer” as used herein refers to a nucleotide sequence that can increase the level of transcription of a nucleic acid encoding a protein and/or RNA molecule of interest, and/or increase or modify the translational efficiency of a transcript following transcription. In some aspects, enhancer sequences (generally 50-1500 bp in length) generally increase the level of transcription by providing additional binding sites for transcription-associated proteins (e.g., transcription factors), and/or stabilize or modify post-transcriptional regulatory machinery. In some aspects, an enhancer sequence is found within an intronic sequence. In some aspects, an enhancer sequence is found in a 3′ and/or 5′ UTR. In some aspects, an enhancer region is found downstream of a coding sequence comprising a transgene and proximal to a poly adenylation sequence. Unlike promoter sequences, enhancer sequences can act at much larger distance away from the transcription start site (e.g., as compared to a promoter). Non-limiting examples of enhancers include a woodchuck hepatitis virus post-transcriptional regulatory element (WPRE), RSV enhancer, a CMV enhancer, and/or a SV40 enhancer.

### Flanking Untranslated Regions, 5′ UTR and 3′ UTR

In some aspects, any of the constructs described herein can include an untranslated region (UTR), such as a 5′ UTR or a 3′ UTR. UTRs of a gene are transcribed but not translated. A 5′ UTR starts at the transcription start site and continues to the start codon but does not include the start codon. A 3′ UTR starts immediately following the stop codon and continues until the transcriptional termination signal. The regulatory and/or control features of a UTR can be incorporated into any of the constructs, particles, polynucleotide constructs, compositions, kits, or methods as described herein to enhance or otherwise modulate the expression of a gene.

Natural 5′ UTRs include a sequence that plays a role in translation initiation. In some aspects, a 5′ UTR can comprise sequences, like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), and the start codon is followed by another “G”. In some aspects, 5′ UTRs also form secondary structures that are involved in elongation factor binding. In some aspects, a 5′ UTR is included in any of the constructs described herein. Non-limiting examples of 5′ UTRs, including those from the following genes: albumin, serum amyloid A, Apolipoprotein A/B/E, transferrin, alpha fetoprotein, erythropoietin, and Factor VIII, can be used to enhance expression of a nucleic acid molecule, such as an mRNA.

3′ UTRs are known to have stretches of adenosines and uridines (in the RNA form) or thymidines (in the DNA form) embedded in them. These AU-rich signatures are particularly prevalent in genes with high rates of turnover. Based on their sequence features and functional properties, the AU-rich elements (AREs) can be separated into three classes (see e.g., Chen et al., Mol. Cell. Biol. 15:5777-5788, 1995; Chen et al., Mol. Cell Biol. 15:2010-2018, 1995, each of which is incorporated herein by reference for the purposes described herein): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif, two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules may lead to HuR binding and thus, stabilization of the message in vivo.

In some aspects, the introduction, removal, or modification of 3′ UTR AREs can be used to modulate the stability of an mRNA encoding a gene of interest. In other aspects, AREs can be removed or mutated to increase the intracellular stability and thus increase translation and production of a protein of interest.

In some aspects, non-ARE sequences may be incorporated into the 5′ or 3′ UTRs. In some aspects, introns or portions of intron sequences may be incorporated into the flanking regions of the polynucleotide constructs in any of the constructs, particles, polynucleotide constructs, compositions, kits, and methods provided herein. Incorporation of intronic sequences may increase protein production as well as mRNA levels.

### Internal Ribosome Entry Sites (IRES)

In some aspects, a construct described herein can include an internal ribosome entry site (IRES). An IRES forms a complex secondary structure that allows translation initiation to occur from any position with an mRNA immediately downstream from where the IRES is located. There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV).

In some aspects, an IRES sequence that is incorporated into a construct described herein is the foot and mouth disease virus (FMDV) 2A sequence. The Foot and Mouth Disease Virus 2A sequence is a small peptide (approximately 18 amino acids in length) that has been shown to mediate the cleavage of polyproteins. The cleavage activity of the 2A sequence has previously been demonstrated in artificial systems including plasmids and gene therapy constructs (e.g., AAV and retroviruses).

In some aspects, an IRES can be utilized in an AAV construct. In some aspects, a construct can include a polynucleotide construct internal ribosome entry site (IRES). In some aspects, an IRES can be part of a composition comprising more than one construct. In some aspects, an IRES is used to produce more than one polypeptide from a single gene transcript.

### Splice Sites

In some aspects, any of the constructs provided herein can include splice donor and/or splice acceptor sequences, which are functional during RNA processing occurring during transcription. In some aspects, splice sites are involved in trans-splicing.

### Polyadenylation Sequences

In some aspects, a construct provided herein can include a polyadenylation (poly(A)) signal sequence. Most nascent eukaryotic mRNAs possess a poly(A) tail at their 3′ end, which is added during a complex process that includes cleavage of the primary transcript and a coupled polyadenylation reaction driven by the poly(A) signal sequence. A poly(A) tail confers mRNA stability and transferability. In some aspects, a poly(A) signal sequence is positioned 3′ to a coding sequence.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. A 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In some aspects, a poly(A) tail is added onto transcripts that contain a specific sequence, e.g., a poly(A) signal. A poly(A) tail and associated proteins aid in protecting mRNA from degradation by exonucleases. Polyadenylation also plays a role in transcription termination, export of the mRNA from the nucleus, and translation. Polyadenylation typically occurs in the nucleus immediately after transcription of DNA into RNA, but also can occur later in the cytoplasm. After transcription has been terminated, an mRNA chain is cleaved through the action of an endonuclease complex associated with RNA polymerase. A cleavage site is usually characterized by the presence of the base sequence AAUAAA near the cleavage site. After the mRNA has been cleaved, adenosine residues are added to the free 3′ end at the cleavage site.

As used herein, a “poly(A) signal sequence” or “polyadenylation signal sequence” is a sequence that triggers the endonuclease cleavage of an mRNA and the addition of a series of adenosines to the 3′ end of the cleaved mRNA.

There are several poly(A) signal sequences that can be used in some aspects, including those derived from bovine growth hormone (bGH), mouse-β-globin, mouse-a-globin, human collagen, polyoma virus, the Herpes simplex virus thymidine kinase gene (HSV TK), IgG heavy-chain gene polyadenylation signal, human growth hormone (hGH), and/or the group consisting of SV40 poly(A) site, such as the SV40 late and early poly(A) site.

In some aspects, the poly(A) signal sequence can be AATAAA. The AATAAA sequence may be substituted with other hexanucleotide sequences with homology to AATAAA and that are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, orAATAAG. In some aspects, a poly(A) signal sequence can be a synthetic polyadenylation site.

### Additional Sequences

In some aspects, constructs of the present disclosure may comprise a 2A element or sequence. In some aspects, constructs of the present disclosure may include one or more cloning sites. In some such aspects, cloning sites may not be fully removed prior to manufacturing for administration to a subject. In some aspects, cloning sites may have functional roles including as linker sequences, or as portions of a Kozak site. As will be appreciated by those skilled in the art, cloning sites may vary significantly in primary sequence while retaining their desired function.

In some aspects, a 2A element is a T2A, P2A, E2A, and/or F2A element. In some aspects, a 2A sequence may comprise an optional 5 linker sequence, such as but not limited to GSG (e.g., Glycine, Serine, Glycine).

### Destabilization Domains

In some aspects, any of the constructs provided herein can optionally include a sequence encoding a destabilizing domain (“a destabilizing sequence”) for temporal and/or spatial control of protein expression. Non-limiting examples of destabilizing sequences include sequences encoding a FK506 sequence, a dihydrofolate reductase (DHFR) sequence, or other exemplary destabilizing sequences.

In the absence of a stabilizing ligand, a protein sequence operatively linked to a destabilizing sequence is degraded by ubiquitination. In contrast, in the presence of a stabilizing ligand, protein degradation is inhibited, thereby allowing the protein sequence operatively linked to the destabilizing sequence to be actively expressed. As a positive control for stabilization of protein expression, protein expression can be detected by conventional means, including enzymatic, radiographic, colorimetric, fluorescence, or other spectrographic assays, fluorescent activating cell sorting (FACS) assays, and/or immunological assays (e.g., enzyme linked immunosorbent assay (ELISA), radioimmunoassay (RIA), and immunohistochemistry).

Additional examples of destabilizing sequences are known in the art. In some aspects, the destabilizing sequence is a FK506- and rapamycin-binding protein (FKBP12) sequence, and the stabilizing ligand is Shield-I (Shld1). In some aspects, a destabilizing sequence is a DHFR sequence, and a stabilizing ligand is trimethoprim (TMP).

### Reporter Sequences or Elements

In some aspects, constructs provided herein can optionally include a sequence encoding a reporter polypeptide and/or protein (“a reporter sequence”). Non-limiting examples of reporter sequences include DNA sequences encoding: a beta-lactamase, a betagalactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with control elements which drive their expression, the reporter sequence can provide signals detectable by conventional means, including enzymatic, radiographic, colorimetric, fluorescence, or other spectrographic assays, fluorescent activating cell sorting (FACS) assays and/or immunological assays (e.g., enzyme linked immunosorbent assay (ELISA), radioimmunoassay (RIA), and immunohistochemistry). In some aspects, a reporter sequence is a FLAG tag (e.g., a 3xFLAG tag), and the presence of a construct carrying the FLAG tag in a cell is detected by protein binding or detection assays (e.g., Western blots, immunohistochemistry, radioimmunoassay (RIA), mass spectrometry).

In some aspects, a reporter sequence is the Lacz gene, and the presence of a construct carrying the Lacz gene in a cell is detected by assays for beta-galactosidase activity. In some aspects, a reporter sequence is a fluorescent protein (e.g., green fluorescent protein (GFP)) or luciferase. In aspects where a reporter sequence is a fluorescent protein or luciferase, the presence of a construct carrying the fluorescent protein or luciferase in a cell may be measured by fluorescent imaging techniques (e.g., fluorescent microscopy or FACS) or light production in a luminometer (e.g., a spectrophotometer or an IVIS imaging instrument). In some aspects, a reporter sequence can be used to verify tissue-specific targeting capabilities and/or tissue-specific promoter regulatory and/or control activity of any of the constructs described herein.

### III. Cells and Method of Transduction or Transfection

In some aspects the current disclosure also encompasses a cell comprising the polynucleotide construct disclosed herein, or a vector, for example a viral vector, as disclosed herein. In some aspects, the cells are from a cell line commonly used to maintain and grow viral vectors and/or maintain polynucleotide constructs. In another aspect, contemplated are the use of host cells into which a polynucleotide construct, vector, or nucleic acid has been introduced. A polynucleotide construct encoding the SLC52A2 protein can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors. Host cells which may be used to maintain and produce disclosed viral vectors and/or the polynucleotide constructs include HEK293, HEK293T, HeLa, Sf9, BHK-21, A549, Vero, CHO, PER.C6. In some aspects, the cell may be a transduced with the viral vector or the polynucleotide construct in vivo (for therapeutic purposes), ex vivo (for example, into patient derived cells) or in vitro for testing, production and/or maintenance.

Viral vectors may be introduced into a desired cell by direct infection, in which viral particles are simply added to the culture medium containing the target cells or administered in vivo. The viral vector binds to specific receptors on the cell surface, leading to entry via endocytosis or membrane fusion, depending on the virus type. Lentivirus, adenovirus, and adeno-associated virus (AAV) all utilize this method. The efficiency of direct transduction may be optimized by changing the multiplicity of infection (MOI): the ratio of viral particles to target cells, which should be carefully adjusted to balance efficiency and toxicity, the incubation time, or inclusion of various chemicals such as polybrene, protamine sulfate, or hexadimethrine bromide which increase viral binding and internalization, particularly for retroviral and lentiviral vectors.

In some aspects, the current disclosure also encompasses a method of transducing a motor neuron with the viral vector disclosed, the method comprising, pre-treating the motor neuron with sialidase; contacting the cell with the viral vector. Pre-treatment with sialidase of RTD motoneurons showed a significant increase in neurites' length when compared to pathological untreated samples demonstrating that AAV9/SLC52A2 gene therapy can rescue RTD motoneurons.

In addition to viral delivery employing the viral vectors mentioned above, several non-viral methods for the transfer of polynucleotide constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the polynucleotide construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement), or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the polynucleotide construct is delivered to a cell and where nucleic acid remains, is dependent on the type of construct employed.

In yet another embodiment, the polynucleotide construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the motor neurons of a subject. This may require parenteral delivery, localized delivery, and/or localized surgical exposure of the tissue or cells. Again, DNA encoding a particular gene may be delivered via this method and still be incorporated by the present disclosure.

### IV. Pharmaceutical Compositions

In some aspects the current disclosure also encompasses a pharmaceutical composition comprising a polynucleotide construct disclosed herein, or a viral vector disclosed herein, or a transgenic cell disclosed herein, and at least one pharmaceutically acceptable excipient.

AAV vectors, comprising the disclosed polynucleotide construct encoding the RT polypeptide of the present invention can be formulated using one or more excipients to: (1) increase stability; (2) increase cell transfection or transduction; (3) permit the sustained or delayed release; or (4) alter the biodistribution (e.g., target the viral vector to specific tissues or cell types such as brain and motor neurons).

Formulations of the present invention can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present invention may be formulated using self-assembled nucleic acid nanoparticles.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some aspects, a pharmaceutically acceptable excipient may be at least 95%, at least 96%, at least 97%, at least 98%, at least 99%, or 100% pure. In some aspects, an excipient is approved for use for humans and for veterinary use. In some aspects, an excipient may be approved by United States Food and Drug Administration. In some aspects, an excipient may be of pharmaceutical grade. In some aspects, an excipient may meet the standards of the United States Pharmacopoeia (USP), the European Pharmacopoeia (EP), the British Pharmacopoeia, and/or the International Pharmacopoeia.

In certain aspects, compositions disclosed herein may compromise one or more pharmaceutically acceptable excipient(s), diluents, and/or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art.

In some aspects, pharmaceutical compositions herein may include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, or any combination thereof. Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In certain aspects, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In some aspects, any of the well-known techniques, carriers, and excipients may be used as suitable and/or as understood in the art.

In certain aspects, pharmaceutical compositions described herein may be an aqueous suspension comprising one or more polymers as suspending agents. In some aspects, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In some aspects, pharmaceutical compositions disclosed herein may comprise at least about 5%, at least about 10%, at least about 20%, at least about 25%, at least about 30%, at least about 35%, at least about 40%, at least about 45%, or at least about 50% total amount of polymers as suspending agent(s) by total weight of the composition. In some aspects, pharmaceutical compositions disclosed herein may comprise about 5% to about 99%, about 10%, about 95%, or about 15% to about 90% total amount of polymers as suspending agent(s) by total weight of the composition.

In certain aspects, pharmaceutical compositions disclosed herein may comprise a viscous formulation. In some aspects, viscosity of composition herein may be increased by the addition of one or more gelling or thickening agents. In some aspects, compositions disclosed herein may comprise one or more gelling or thickening agents in an amount to provide a sufficiently viscous formulation to remain on treated tissue. In some aspects, pharmaceutical compositions disclosed herein may comprise at least about 5%, at least about 10%, at least about 20%, at least about 25%, at least about 30%, at least about 35%, at least about 40%, at least about 45%, or at least about 50% total amount of gelling or thickening agent(s) by total weight of the composition. In some aspects, pharmaceutical compositions disclosed herein may comprise about 5% to about 99%, about 10%, about 95%, or about 15% to about 90% total amount of gelling or thickening agent(s) by total weight of the composition. In some aspects, suitable thickening agents for use herein can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other aspects, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda (dextrose, maltodextrin and sucralose), or any combination thereof.

In certain aspects, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some aspects, pharmaceutical compositions disclosed herein may comprise at least about 5%, at least about 10%, at least about 20%, at least about 25%, at least about 30%, at least about 35%, at least about 40%, at least about 45%, or at least about 50% total amount of one or more agents by total weight of the composition. In some aspects, pharmaceutical compositions disclosed herein may comprise about 5% to about 99%, about 10%, about 95%, or about 15% to about 90% total amount of one or more agents by total weight of the composition. In some aspects, one or more of these agents may be added to improve the performance, efficacy, safety, shelf-life and/or other property of the muscarinic antagonist composition of the present disclosure. In some aspects, additives may be biocompatible, without being harsh, abrasive, and/or allergenic.

In certain aspects, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some aspects, any pharmaceutically acceptable organic or inorganic acid may be used. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more acidifying agents by total weight of the composition. In some aspects, pharmaceutical compositions disclosed herein may comprise about 5% to about 99%, about 10%, about 95%, or about 15% to about 90% total amount of one or more acidifying agents by total weight of the composition.

In certain aspects, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some aspects, any pharmaceutically acceptable organic or inorganic base can be used. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more alkalizing agents by total weight of the composition. In some aspects, pharmaceutical compositions disclosed herein may comprise about 5% to about 99%, about 10%, about 95%, or about 15% to about 90% total amount of one or more alkalizing agents by total weight of the composition.

In certain aspects, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate, sodium metabisulfite and other materials known to one of ordinary skill in the art. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more antioxidants by total weight of the composition. In some aspects, pharmaceutical compositions disclosed herein may comprise about 5% to about 99%, about 10%, about 95%, or about 15% to about 90% total amount of one or more antioxidants by total weight of the composition.

In certain aspects, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some aspects, any pharmaceutically acceptable organic or inorganic buffer can be used. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more buffering agents by total weight of the composition. In some aspects, pharmaceutical compositions disclosed herein may comprise about 5% to about 99%, about 10%, about 95%, or about 15% to about 90% total amount of one or more buffering agents by total weight of the composition.

In some aspects, the amount of one or more buffering agents may depend on the desired pH level of a composition. In some aspects, pharmaceutical compositions disclosed herein may have a pH of about 6 to about 9. In some aspects, pharmaceutical compositions disclosed herein may have a pH greater than about 8, greater than about 7.5, greater than about 7, greater than about 6.5, or greater than about 6.

In certain aspects, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some aspects, any pharmaceutically acceptable preservative can be used. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more preservatives by total weight of the composition. In some aspects, pharmaceutical compositions disclosed herein may comprise about 5% to about 99%, about 10%, about 95%, or about 15% to about 90% total amount of one or more preservatives by total weight of the composition.

In certain aspects, pharmaceutical compositions disclosed herein may comprise one or more surface-acting reagents or detergents. In some aspects, surface-acting reagents or detergents may be synthetic, natural, or semi-synthetic. In some aspects, compositions disclosed herein may comprise anionic detergents, cationic detergents, zwitterionic detergents, ampholytic detergents, amphoteric detergents, nonionic detergents having a steroid skeleton, or a combination thereof. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more surface-acting reagents or detergents by total weight of the composition. In some aspects, pharmaceutical compositions disclosed herein may comprise about 5% to about 99%, about 10%, about 95%, or about 15% to about 90% total amount of one or more surface-acting reagents or detergents by total weight of the composition.

In certain aspects, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate, and sodium saccharin and others known to those of ordinary skill in the art. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more stabilizers by total weight of the composition. In some aspects, pharmaceutical compositions disclosed herein may comprise about 5% to about 99%, about 10%, about 95%, or about 15% to about 90% total amount of one or more stabilizers by total weight of the composition.

In some aspects, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In some aspects, a vapor pressure depression method is used to calculate the osmolarity of the compositions disclosed herein. In some aspects, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In some aspects, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some aspects, a pharmaceutical composition described herein may have an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In some aspects, pharmaceutical compositions disclosed herein may comprise at least about 5%, at least about 10%, at least about 20%, at least about 25%, at least about 30%, at least about 35%, at least about 40%, at least about 45%, at least about 50% total amount of one or more tonicity modifiers by total weight of the composition. In some aspects, pharmaceutical compositions disclosed herein may comprise about 5% to about 99%, about 10%, about 95%, or about 15% to about 90% total amount of one or more tonicity modifiers by total weight of the composition.

### Dosage Formulations

In certain aspects, the present disclosure provides compositions comprising one or more inhibitors disclosed herein, formulated for one or more routes of administration. Suitable routes of administration may, for example, include intravenous, intracranial, intrathecal, subcutaneous, intranasal route, cranial, transmucosal, trans-nasal, transcranial, intracerebroventricular, intestinal, and/or parenteral delivery. In some aspects, compositions herein formulated can be formulated for parenteral delivery. In some aspects, compositions herein formulated can be formulated intramuscular, subcutaneous, intramedullary, intravenous, intraperitoneal, intracranial intrathecal, and/or intranasal injections.

In certain aspects, one may administer a composition herein in a local or systemic manner, for example, via local injection of the pharmaceutical composition directly into a tissue region of a patient. In some aspects, a pharmaceutical composition disclosed herein can be administered parenterally, e.g., by intravenous injection, intracerebroventricular injection, intra-cisterna magna injection, intra-parenchymal injection, or a combination thereof. In some aspects, a pharmaceutical composition disclosed herein can be administered to subject as disclosed herein. In some aspects, a pharmaceutical composition disclosed herein can administered to human patient. In some aspects, a pharmaceutical composition disclosed herein can be administered to a human patient via at least two administration routes. In some aspects, the combination of administration routes by be intracerebroventricular injection and intravenous injection; intrathecal injection and intravenous injection; intra-cisterna magna injection and intravenous injection; and/or intra-parenchymal injection and intravenous injection.

In certain aspects, pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

In certain aspects, pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen. For injection, the active ingredients of a pharmaceutical composition herein may be formulated in aqueous solutions, preferably in physiologically compatible buffers such as Hank's solution, Ringer's solution, physiological salt buffer, or any combination thereof.

In certain aspects, pharmaceutical compositions described herein may be formulated in the form of a nanoparticle. The nanoparticle may have a monolayer enclosing the nanoparticle core, wherein the polynucleotide construct is disposed within the nanoparticle core. In an aspect, the nanoparticle core includes a solid lipid (i.e., lipid that remains solid at room temperature and body temperature) or a liquid lipid (i.e., oil, which remains liquid at room temperature and body temperature, for example, vegetable oil or a lipid extracted from human adipose tissue). In particular, aspects of the present disclosure include nanoparticles and compositions for the controlled and/or sustained release (e.g., release at a predetermined rate to maintain a certain concentration for a certain period of time) of an agent, such as a polynucleotide construct from the nanoparticle.

In certain aspects, pharmaceutical compositions described herein may be formulated for parenteral administration, e.g., by bolus injection or continuous infusion. Formulations for injection herein may be presented in unit dosage form, e.g., in ampoules or in multidose containers with optionally, an added preservative. In some aspects, compositions herein may be suspensions, solutions or emulsions in oily or aqueous vehicles, and/or may contain formulatory agents such as suspending, stabilizing and/or dispersing agents.

In certain aspects, pharmaceutical compositions herein formulated for parenteral administration may include aqueous solutions of the active preparation (e.g., a polynucleotide construct) in water-soluble form. In some aspects, compositions herein comprising suspensions of the active preparation may be prepared as oily or water-based injection suspensions. Suitable lipophilic solvents and/or vehicles for use herein may include, but are not limited to, fatty oils such as sesame oil, or synthetic fatty acids esters such as ethyl oleate, triglycerides or liposomes. In some aspects, compositions herein comprising aqueous injection suspensions may contain substances which increase the viscosity of the suspension, such as sodium carboxymethyl cellulose, sorbitol, and/or dextran. In some aspects, compositions herein comprising a suspension may also contain one or more suitable stabilizers and/or agents which increase the solubility of the active ingredients (e.g., a polynucleotide construct/vector molecule) to allow for the preparation of highly concentrated solutions.

In some aspects, compositions herein may comprise the active ingredient in a powder form for constitution with a suitable vehicle, e.g., sterile, pyrogen-free water-based solution, before use.

Pharmaceutical compositions suitable for use in context of the present disclosure may include compositions wherein the active ingredients can be contained in an amount effective to achieve the intended purpose. In some aspects, a therapeutically effective amount means an amount of active ingredients (e.g., a polynucleotide construct molecule) effective to prevent, slow, alleviate or ameliorate symptoms of a disorder (e.g., EDE) or prolong the survival of the subject being treated. Determination of a therapeutically effective amount is well within the capability of those skilled in the art, especially in light of the detailed disclosure provided herein. For any preparation used in the methods of the present disclosure, the therapeutically effective amount or dose can be estimated initially from in vitro and cell culture assays and/or screening platforms disclosed herein. For example, a dose can be formulated in animal models to achieve a desired concentration or titer. Such information can be used to more accurately determine useful doses in humans.

In some aspects, toxicity and therapeutic efficacy of the active ingredients disclosed herein (e.g., a polynucleotide construct/vector) can be determined by standard pharmaceutical procedures in vitro, in cell cultures or experimental animals. In some aspects, data obtained from these in vitro and cell culture assays and animal studies can be used in formulating a range of dosage for use in a human subject. In some aspects, a dosage for use herein may vary depending upon the dosage form employed and the route of administration utilized. The exact formulation, route of administration and dosage can be chosen by the individual physician in view of the patient's condition.

In certain aspects, dosage amounts and/or dosing intervals may be adjusted individually to brain or blood levels of the active ingredient that are sufficient to induce or suppress the biological effect (minimal effective concentration, MEC). In some aspects, the MEC for an active ingredient (e.g., a polynucleotide construct/vector molecule or composition disclosed herein) may vary for each preparation but can be estimated from in vitro data. In some aspects, dosages necessary to achieve the MEC herein may depend on individual characteristics and route of administration. Detection assays can be used to determine plasma concentrations.

In certain aspects, depending on the severity and responsiveness of the condition to be treated, dosing with compositions herein can be of a single or a plurality of administrations, with course of treatment lasting from several days to several weeks or until cure is effected or diminution of the disease state is achieved.

In certain aspects, amounts of a composition herein to be administered will be dependent on the subject being treated, the severity of the affliction, the manner of administration, the judgment of the prescribing physician, and the like. In some aspects, effective doses may be extrapolated from dose-responsive curves derived from in vitro or in vivo test systems.

According to the present invention, the vector, e.g., AAV vector, comprising the disclosed nucleic acid sequence may be formulated for CNS delivery. Agents that cross the brain blood barrier may be used.

The active ingredient may be administered at once or may be divided into a number of smaller doses to be administered at intervals of time. The precise dosage and duration of treatment is a function of the disease being treated and may be determined empirically using known testing protocols or by extrapolation from in vivo or in vitro test data. Concentrations and dosage values may also vary with the severity of the condition to be alleviated. For any particular subject, specific dosage regimens can be adjusted overtime according to the individual need and the professional judgment of the person administering or supervising the administration of the compositions, and that the concentration ranges set forth herein are exemplary only and are not intended to limit the scope or practice of the claimed compositions. The compositions of the disclosure may comprise an AAV alone, or in combination with one or more other viruses (e.g., a second AAV encoding having one or more different transgenes). In some aspects, a composition comprises 1, 2, 3, 4, 5, 6, 7, 8, 9, 10, or more different AAVs each having one or more different transgenes.

The AAVs are administered in sufficient amounts to transfect the cells of a desired tissue and to provide sufficient levels of gene transfer and expression without undue adverse effects.

The dose of AAV virions required to achieve a particular “therapeutic effect,” e.g., the units of dose in genome copies/per kilogram of body weight (GC/kg), will vary based on several factors including, but not limited to: the route of AAV virion administration, the level of gene or RNA expression required to achieve a therapeutic effect, the specific disease or disorder being treated, and the stability of the gene or RNA product. One of skill in the art can readily determine a AAV virion dose range to treat a patient having a particular disease or disorder based on the aforementioned factors, as well as other factors that are well known in the art.

An effective amount of an AAV is an amount sufficient to infect an animal, and/or target a desired tissue. In some aspects, an effective amount of an AAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the AAV is generally in the range of from about 1 ml to about 100 ml of solution containing from about 10⁹ to 10¹⁶ genome copies. In some cases, a dosage between about 10¹¹ to 10¹³ AAV genome copies is appropriate. In certain aspects, 10¹² or 10¹³ AAV genome copies is effective to target CNS tissue. In some cases, stable transgenic animals are produced by multiple doses of an AAV.

In some aspects, a dose of AAV is administered to a subject no more than once per calendar day (e.g., a 24-hour period). In some aspects, a dose of AAV is administered to a subject no more than once per 2, 3, 4, 5, 6, or 7 calendar days. In some aspects, a dose of AAV is administered to a subject no more than once per calendar week (e.g., 7 calendar days). In some aspects, a dose of AAV is administered to a subject no more than bi-weekly (e.g., once in a two calendar week period). In some aspects, a dose of AAV is administered to a subject no more than once per calendar month (e.g., once in 30 calendar days). In some aspects, a dose of AAV is administered to a subject no more than once per six calendar months. In some aspects, a dose of AAV is administered to a subject no more than once per calendar year (e.g., 365 days or 366 days in a leap year).

In some aspects, AAV compositions are formulated to reduce aggregation of AAV particles in the composition, particularly where high AAV concentrations are present. Methods for reducing aggregation of AAVs are well known in the art and include, for example, addition of surfactants, pH adjustment, salt concentration adjustment, etc.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### V. Methods of Treatment

In some aspects, the current disclosure also encompasses a method of treating a disease or disorder in a subject in need thereof, the method comprising administering to the subject an effective amount of the pharmaceutical composition disclosed herein.

In some aspects, the subject has a defective SLC52A2 gene. In some aspects, the subject has or is suspected of having riboflavin transporter deficiency syndrome (RTD). Suitable subjects may include, without limit, humans, as well as companion animals such as cats, dogs, rodents, and horses; research animals such as rabbits, sheep, pigs, dogs, primates, mice, rats, and other rodents; agricultural animals such as cows, cattle, pigs, goats, sheep, horses, deer, chickens, and other fowl; zoo animals; and primates such as chimpanzees, monkeys, and gorillas. The subject can be of any age without limitation. In an aspect, the subject may be a human.

Generally, the composition will be administered in a therapeutically effective amount which includes prophylactic amounts or lower dosages for example, when combined with another agent. As used herein, “an effective amount” refers to doses of compound sufficient to provide circulating or local concentrations high enough to impart a beneficial effect on the recipient thereof. The precise amount to be administered can be determined by the skilled practitioner in view of desired dosages, side effects, and medical history of the patient.

In various aspects, the compositions may be administered intrathecally.

### VI. Kits

Some aspects of the present disclosure include kits for packaging and transporting any agent disclosed herein and further include at least one container.

In some aspects, the kit can additionally comprise instructions for use of an agent in any of the methods described herein. The included instructions may comprise a description of administration of pharmaceutical compositions as disclosed herein to a subject to achieve the intended activity in a subject. The kit may further comprise a description of selecting a subject suitable for treatment based on identifying whether the subject is in need of the treatment. In some aspects, the instructions may comprise a description of administering pharmaceutical compositions disclosed herein to a subject in need thereof.

### EXAMPLES

The following examples are included to demonstrate certain aspects of the disclosure. It should be appreciated by those of skill in the art that the techniques disclosed in the examples which follow represent techniques discovered by the inventors to function well in the practice of the disclosure and thus can be considered to constitute certain modes for its practice. However, those of skill in the art should, in light of the present disclosure, appreciate that many changes can be made in the specific aspects which are disclosed and still obtain a like or similar result without departing from the spirit and scope of the inventions described herein.

Riboflavin transporter deficiency syndrome (RTD) is a rare childhood-onset neurodegenerative disorder caused by mutations in SLC52A2 and SLC52A3 genes, encoding the riboflavin (RF) transporters hRFVT2 and hRFVT3. In some aspects, the current disclosure pertains to gene therapies for RTD Type 2, which is caused due to variants in the SLC52A2 gene. There is no cure for RTD patients and, although studies have reported clinical improvements with administration of RF, an effective treatment is still unavailable. In some aspects, the current disclosure is based on development of a human codon optimized version of the SLC52A2 gene. In some aspects, the current disclosure is based on extensive experimentation to test gene replacement therapy on RTD type 2 patient-derived motoneurons incorporating the codon optimized version of SLC52A2 in an adeno-associated viral vector 2/9 (AAV9). In vitro transduction of motoneurons using Sialidase treatment was optimized. Treated RTD motoneurons showed a significant increase in neurites' length when compared to pathological untreated samples demonstrating that AAV9-SLC52A2 gene therapy can rescue RTD motoneurons. This leads the path towards in vivo studies offering a potential treatment for RTD patients.

### Example 1: AAV9-SLC52A2 Design

The AAV9-GFP vector design has been previously described (Gray et al. 2011). The scAAV9/UsP-hSLC52A2opt-BGHpA (AAV9-SLC52A2) vector was developed similarly, but with a moderate strong UsP promoter and bovine growth hormone (BGH) polyadenylation signal to drive expression of a codon-optimized human SLC52A2 sequence. Codon optimization was carried out by ATUM (Menlo Park, CA, USA). Both AAV vectors used a self-complementary genome configuration. The AAV vectors were manufactured by the University of North Carolina Vector core, according to published methods.

### Example 2: Gene Therapy Successfully Rescues RTD Motoneurons Neurites' Length

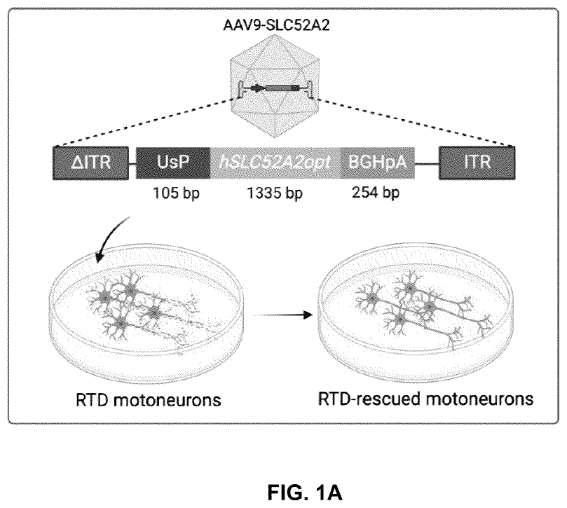

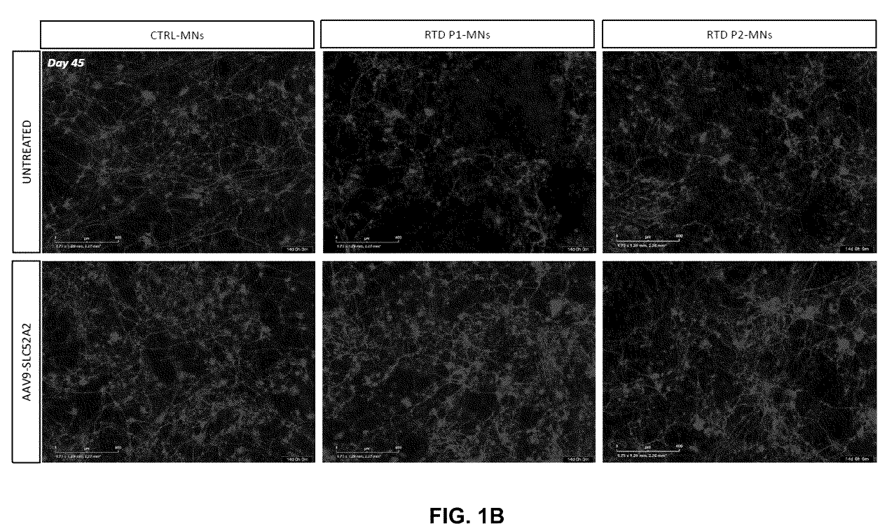

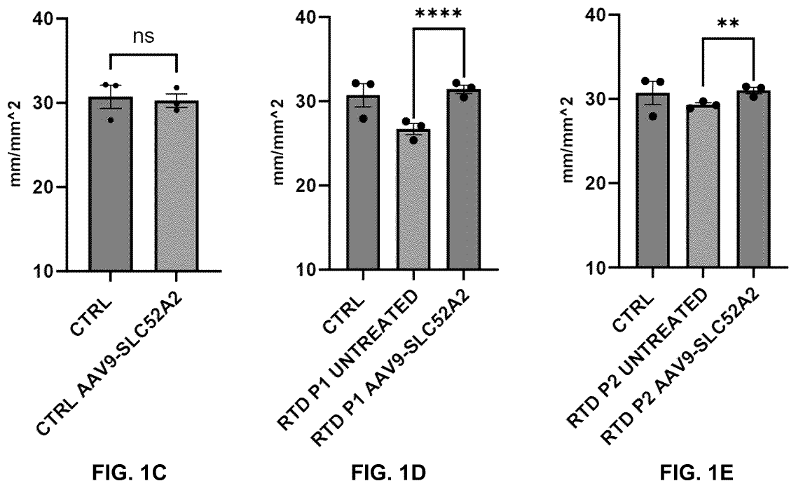

To treat RTD motoneurons, a scAAV9/UsP-hSLC52A2opt-BGHpA (AAV9/SLC52A2) vector carrying the human codon optimized SLC52A2 cDNA was used (FIG. 1A).

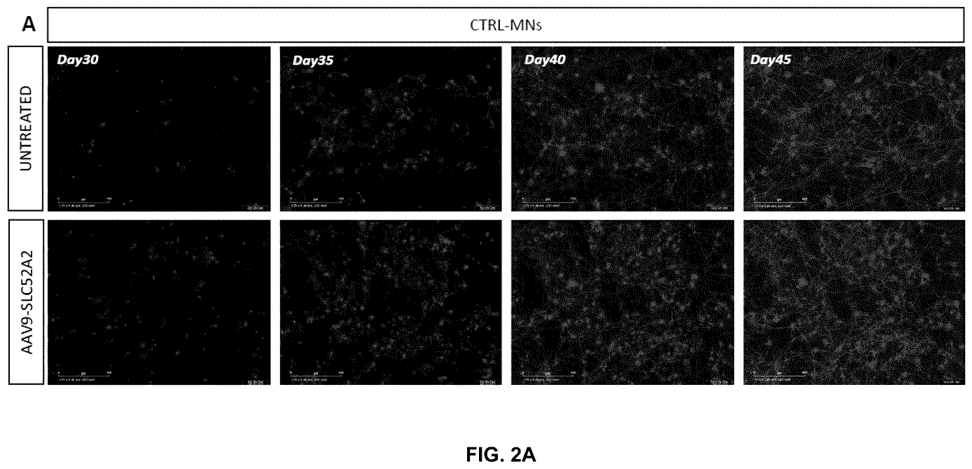

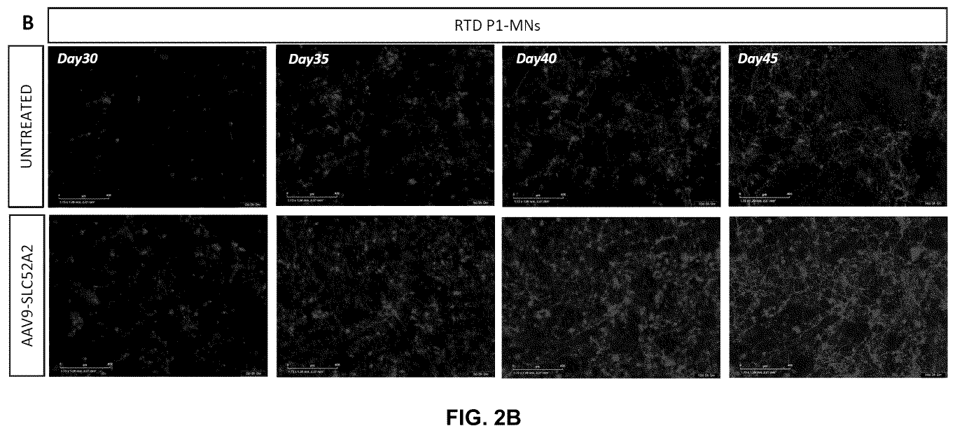

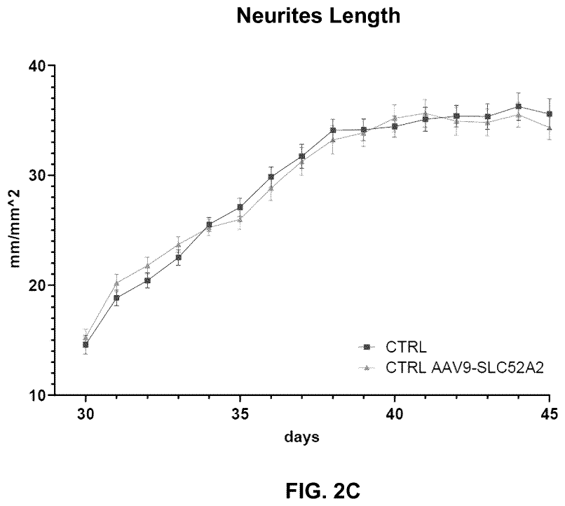

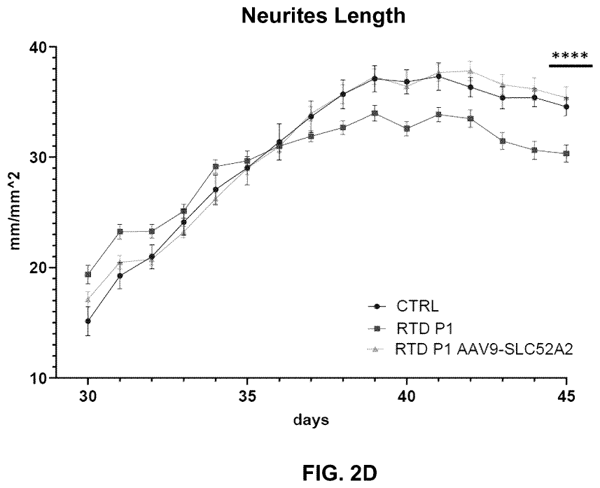

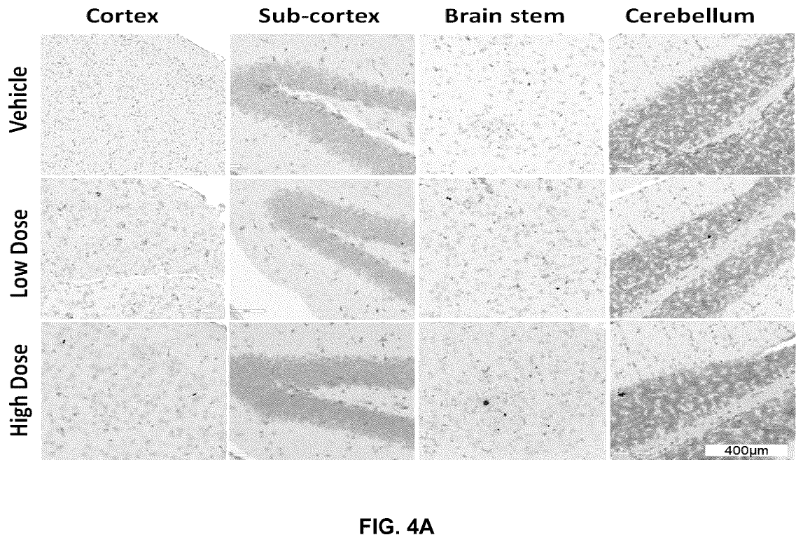

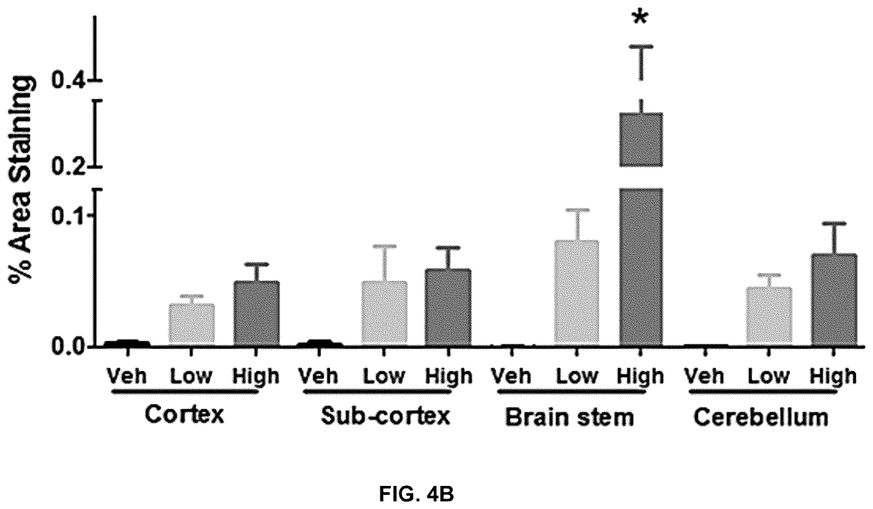

RTD P1 and P2 motoneurons were transduced with the AAV9/SLC52A2 at day 25 in culture using the optimized 1.25 u/ml sialidase treatment and m.o.i of 107 and evaluated motoneurons at day 45 in culture. To establish the effects of the gene therapy on the neurites' length disease phenotype motoneurons were also transduced with the Neurolight red lentivirus (FIG. 1B) for the automated measurement of the neurites' length). Importantly, following AAV treatment, it was found that neurites' length of RTD P1 and P2 motoneurons were significantly longer than untreated (FIG. 4C-4E, p=0.5011 for CTRL, **p=0.0050 for RTD P1, *p=0.0192 for RTD P2, ANOVA; mean±SEM; n=12 images, N=3 independent experiments). Furthermore, length was restored to levels similar to control MNs (FIGS. 1C-1E). A more pronounced rescue was observed in treated RTD P1 motoneurons, therefore to evaluate the effect of the gene therapy on neurogenesis, untreated and treated control and RTD P1 motoneurons were imaged at several time points (days 30, 35, 40 and 45 of neuronal differentiation) (FIGS. 2A, 2B). It was seen that in untreated RTD motoneurons, a presumptive neural network was able to form, but over time, motoneurons neurites were lost and cells underwent neurodegeneration (FIGS. 2A-2B). Meanwhile, following gene therapy with the AAV9-SLC52A2 vector the neural network in RTD patient was preserved over time, showing a progressive amelioration of the neurites' length in transduced RTD motoneurons (FIG. 2C, **p=0.001, ANOVA; mean±SEM; n=12 images, N=3 independent experiments). No changes in terms of neurites' length nor network formation/degeneration were observed in control motoneurons treated with AAV9-UsP-SLC52A2 indicating the overexpression of SLC52A2 is not detrimental to neurons in vitro (FIG. 2D).

### Example 3: Conclusion from Examples 1 and 2

In vitro stem cell-derived motoneurons are a valuable tool to analyze the pathomechanisms underlying motoneuron diseases. One of the most compromised features in RTD motoneurons is the cytoskeleton. The morphometric analyses of RTD iPSC-derived motoneurons provided here confirmed that RTD disease leads to neurites that are significantly shorter than those of the healthy motoneurons.

Promisingly, gene therapy was able to restore neurites' length of RTD motoneurons generating long neurites maintaining a robust neural network during the neurogenesis compared to the untreated RTD motoneurons, which resulted in a breakable and fragile neuronal network undergoing degradation over time.

Collectively, these results indicate that AAV9/SLC52A2 vector rescues the neural phenotype in motoneurons derived from RTD Type 2 patient iPSCs and provides gene therapy as a potential clinical treatment for these patients. Currently, many research efforts are focused on finding new therapeutic strategies that would treat RTD independently from patients' variants. These findings, offer the first insights into gene therapy efficacy for RTD patients worldwide.

### Example 4: Toxicity and Gene Expression Efficiency of AAV9/SLC52A2 in Wild Type (WT) C57BL/6J Mice

The objective of the examples provided below was to characterize the toxicity and gene expression of AAV9/SLC52A2ollowing a single intrathecal (IT) injection in wild type (WT) C57BL/6J mice. As provided herein, the AAV9/SLC52A2 is being developed for treatment of Riboflavin Transporter Deficiency (RTD), but may be used for any RTD linked disease or disorder.

### Example 5: Materials and Methods

Plasmid Design and Development

Plasmid was designed essentially as provided above. Briefly a plasmid was designed and developed containing the transgene of a human SLC52A2 codon-optimized construct (hSLC52A2opt). The DNA sequence was modified such that the final amino acid sequence is unchanged, but the transgene sequence is distinct from the endogenous gene to enable specific transgene detection by various molecular methods. The transgene consists of a human SLC52A2 DNA coding sequence between a promoter and a polyadenylation signal. Sanger sequencing was used to confirm the plasmid sequence.

scAAV9/SLC52A2 Vector Preparation

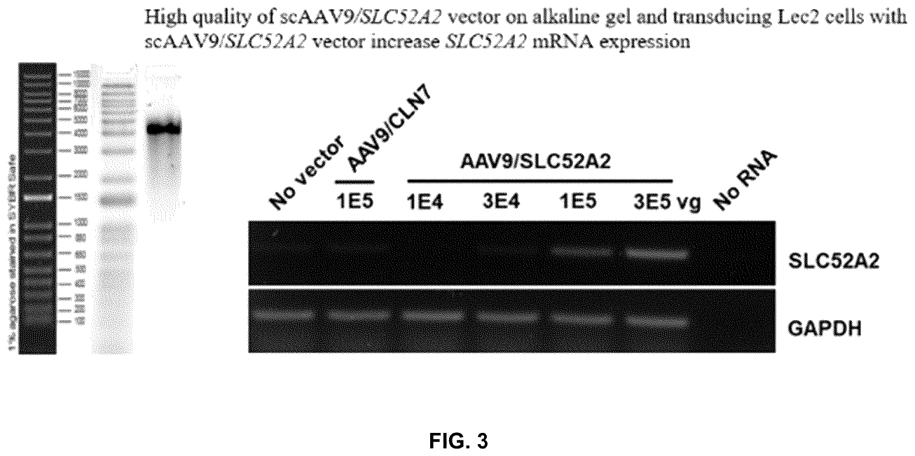

The established plasmid was packaged into scAAV9 vectors (Gray et al. 2011) which are 10-100 times more efficient at transduction compared to traditional single-stranded (ss) AAV vectors. The scAAV9 vectors were produced by UNC-VC. The final research-grade product was dialyzed in phosphate-buffered saline (PBS) with additional 212 mM NaCl and 5% D-sorbitol, titered by qPCR, and confirmed by silver stain. RT-qPCR was used to confirm mRNA expression of the transgene in Lec2 cells (FIG. 3).

### Study Design

WT C57BL/6J mice from Jackson Laboratories were assigned to the study as indicated in Table 1.

TABLE 1
Summary of the non-GLP cohorts

| | | | Body weight (g)** | | | Endpoint |
|---|---|---|---|---|---|---|
| | | Age | Male | Female | Dose | (12 months |
| GRP | Route* | (wks) | (n = 10) | (n = 10) | (vg/mouse) | post injection) |
| 1 | IT | 7 | 21.9 ± 0.3 | 16.3 ± 0.5 | Vehicle | Body weights, |
| 2 | | | 21.3 ± 0.3 | 17.3 ± 0.5 | 1.5 × 10¹¹ | Clinical signs, |
| | | | | | (Low) | Adverse events, |
| 3 | | | 20.7 ± 0.4 | 16.1 ± 0.4 | 6 × 10¹¹ | Mortality, and |
| | | | | | (High) | Histopathology |

IT = intrathecal;
vg = vector genome
*IT injections were via lumbar puncture, a 5 μL dose in vehicle (350 mM phosphate-buffered saline, 5% sorbitol).
**Mean ± standard error

The non-GLP studies presented in Table 1 were designed to identify any long-term safety issues of the experimental therapy. The mice were randomized to different groups and injected IT with 5 μL of vehicle or different doses of AAV9/SLC52A2 vectors. The AAV9/SLC52A2 vectors were made by UNC-VC (University of North Carolina —Vector Core).

Mice were monitored for changes in body weight, clinical signs, adverse events, and mortality following the treatment. All mice were weighed weekly for the first month and then monthly thereafter. Any clinical signs or adverse events including neurological symptoms were investigated, evaluated, and recorded. Appropriate supportive or therapeutic interventions were offered per Institutional Animal Care and Use Committee (IACUC) and veterinary guidance. One-month post injection, 6 mice (3 males and 3 females) from each group were euthanized. Mouse brains were used for determining SLC52A2 mRNA expression by RNAscope and mouse serum were used to check serum toxicity panel including aspartate transaminase (AST), total bilirubin (TBIL), albumin (ALB), creatine kinase (CK), and blood urea nitrogen (BUN). Blood and tissue samples were collected from mice that were euthanized for humane reasons. Where possible, a detailed necropsy was performed to investigate or identify the reason for the ailment by a trained technician or veterinary staff. Terminal serum and tissue samples at 12 months following the treatment were collected for serum toxicity panel and histopathological assessment, respectively. The final histopathological evaluation on collected tissue samples was also conducted.

Quantitative data were tested for normal distribution (Shapiro-Wilk normality test) and homogeneity of variance (Brown-Forsythe test). Data sets that passed these two tests were analyzed using one-way ANOVA with a set at 0.05 with Holm-Sidak correction for multiple comparisons. Data sets that did not pass tests for normality or homogeneity of variance were analyzed using Kruskal-Wallis test with a set at 0.05. All pair-wise comparisons were made, with Dunn's correction for multiple comparisons.

### Example 6: Intrathecal (IT) Injection of AAV9/SLC52A2 Vector Dose-Dependently Increased hSLC52A2Opt mRNA in all Brain Regions

High (6×10¹¹ vg/mouse) or low (1.5×10¹¹ vg/mouse) dose of AAV9/SLC52A2 vector was administered IT to WT mice of 7 weeks old. Animals receiving AAV9/SLC52A2 had detectable levels of hSLC52A2opt mRNA in all brain regions assessed (FIG. 4A). The high dose group had significantly higher mRNA levels in brainstem than control animals. While the low dose animals did have detectable levels of mRNA, these levels were not significantly higher than the controls (FIG. 4B).

### Example 7: IT AAV9/SLC52A2 Caused No Elevation of Serum Toxicity Panel

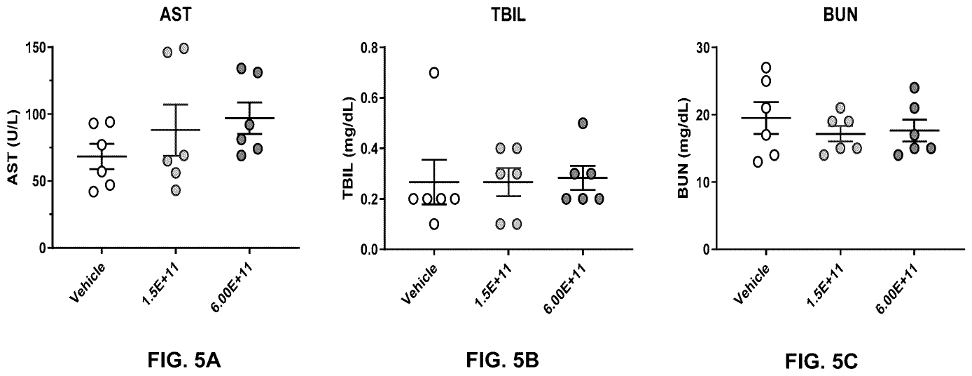

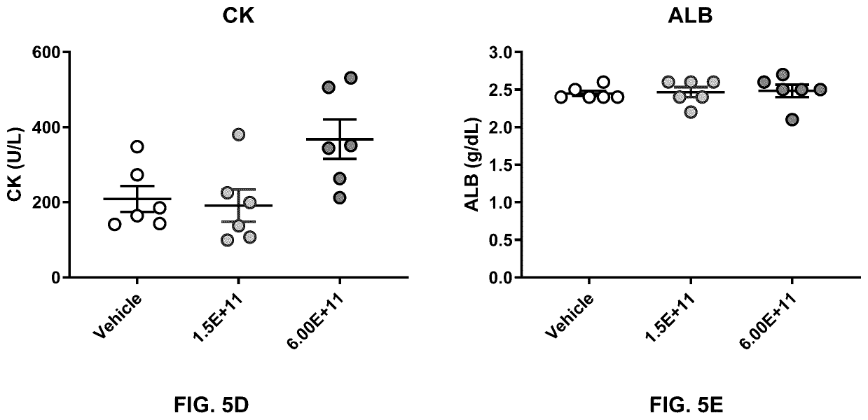

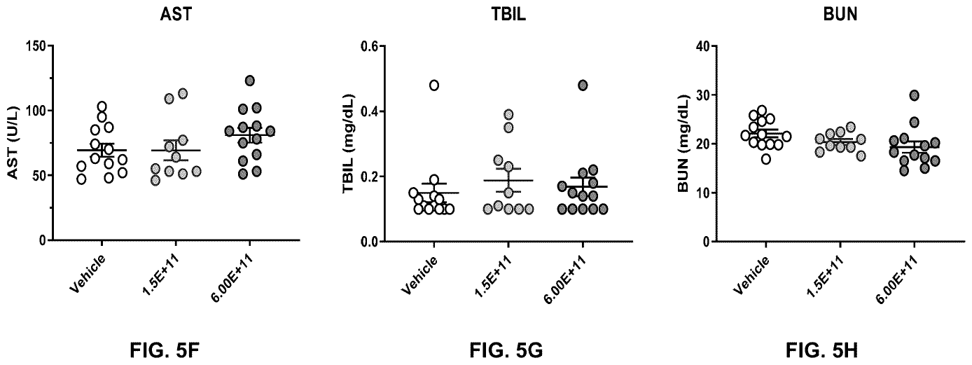

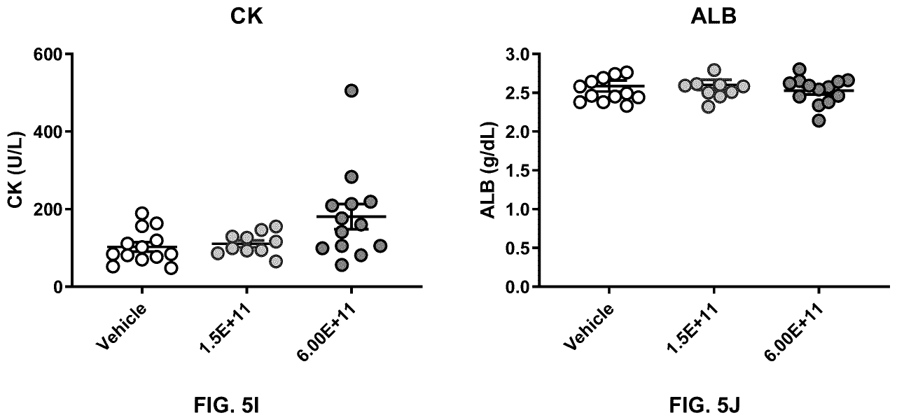

At 1-month post injection, mouse serum was collected for serum chemistry. Animals receiving AAV9/SLC52A2 had normal levels of serum toxicity panel including AS, TBIL, ALB, CK, and BUN 1-month post injection (FIGS. 5A-5E) as well as 12-month post injection (FIGS. 5F-5J), indicating no acute or chronic toxicity to vital organs liver, heart, or kidney by IT AAV9/SLC52A2 vector.

### Example 8: IT AAV9/SLC52A2 Caused No Effects on Body Weight in Male or Female Mice

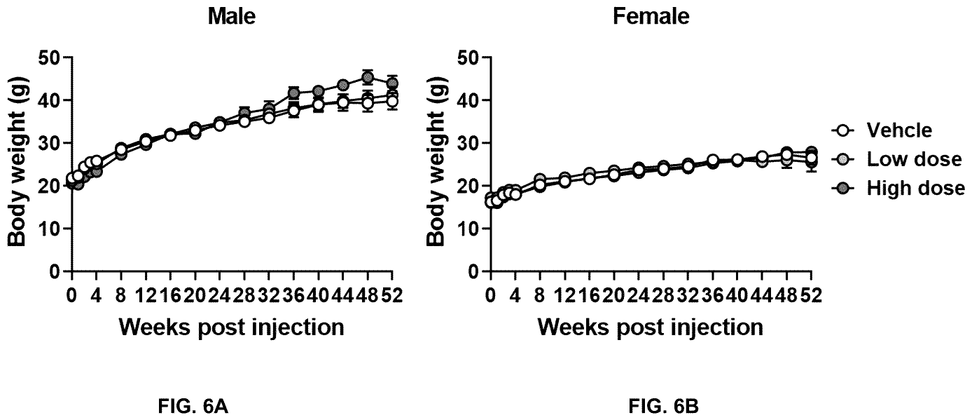

Mice were weighed weekly for the first month following treatment and then monthly thereafter. Body weight was monitored to assess the overall health of the animals. There was no significant difference in body weight between groups within male or female mice at any point of assessment (FIGS. 6A and 6B), suggesting that doses up to 6×10¹¹ vg/mouse are well tolerated in the WT C57BL/6J mice up to 12 months following the treatment.

### Example 9: IT AAV9/SLC52A2 Caused No Death in WT Mice

There were no obvious clinical signs of morbidity in the adult WT mice dosed with AAV9/SLC52A2 at doses up to 6×10¹¹ vg/mouse (Table 1). Six mice in total did not survive to the end of the study, however none of the deaths were treatment related. One female mouse from vehicle group was excluded at 1-week post injection due to hindlimb paralysis by the IT procedure. Three female mice from low dose group were found dead in the same cage due to accidental no water supply at 6 weeks post injection. One male mouse from high dose group was euthanized due to fighting induced severe trauma and tendon exposure on the tail at 33 weeks post injection. One male mouse from low dose group was euthanized due to ulcerative dermatitis around the base of the tail at 48 weeks post injection. There was no other unexpected death in this study over one-year post administration of AAV9/SLC52A2. 36 mice survived to the end of the experiment, further indicating that doses up to 6×10¹¹ vg/mouse are well tolerated in WT C57BL/6J mice up to 12 months following the treatment.

### Example 10: IT AAV9/SLC52A2 Caused No Clinical Signs or Adverse Events in WT Mice

No outward signs of toxicity were noted over the duration of the study. At the end of the experiment, all 36 survival mice were anesthetized and perfused with phosphate-buffered saline containing 1 U/mL heparin and main tissues/organs were harvested. No obvious abnormalities were noticed. All tissues/organs collected were fixed in 10% formalin for 24 hours and then transferred to 70% ethanol. These tissues/organs were sent out for histopathology.

Tissues were fixed in 10% neutral buffered formalin for 1 day, stored in 70% ethanol, trimmed into tissue cassettes. Hematoxylin and Eosin-stained slides were produced from the cassettes. Tissues and the corresponding slides were labeled with the following ID's: WT-61, WT-62, WT-64, WT-65, WT-68, WT-69, WT-70, WT-72, WT-74, WT-75, WT-78, WT-79, WT-80, WT-81, WT-82, WT-85, WT-88, WT-89, WT-90, WT-91, WT-92, WT-95, WT-98, WT-99, WT-100, WT-101, WT-102, WT-104, WT-105, WT-111, WT-112, WT-114, WT-115, WT-118, WT-119, and WT-120. Brain, heart, tricep muscle, liver, lung, gonad, spleen, kidney, sciatic nerve, cervical and lumbar spinal cord were submitted for all animals except for the following instances. The lung was not present for WT-119. There was no ovary present for WT-98, WT-99 and WT-102. These three animals had small sections of uterus or oviduct submitted, which were microscopically normal. There was no gonad or section of reproductive tract present for WT-91, WT-92, WT-101 and WT-118. The triceps muscle was not present for WT-95, WT-102 and WT-114. The sciatic nerve was not present for animal ID WT-68, WT-74, WT-75, WT-80, WT-81, WT-89, WT-90, WT-91, WT-98 and WT-102. The spleen was not present for WT-61.

The cerebrum, cerebellum, and olfactory bulb of all the mice were microscopically normal. The sections of cervical and lumbar cord were present and microscopically normal is all submitted sections except for the following instances. There was no cord present in the lumbar vertebrae of WT-75. There was only one section of cord in WT-80. It was a lumbar section and microscopically normal. The lumbar sections from WT-100, WT-101, WT-114, and WT-118 were from the cauda equina region of the cord and were microscopically normal. Animal WT-70 had focal perivascular infiltrates with a few lymphocytes. These infiltrates can occur spontaneously and are considered an incidental finding.

The sciatic nerves of mouse ID WT-61, WT-64, WT-65, WT-70, WT-72, WT-79, WT-85, WT-92, WT-95, WT-99, WT-100, WT-104, WT-112, WT-114, and WT-118 contained no microscopic abnormalities. There were a few scattered mast cells present in the sciatic nerve of these mice: WT-62, WT-69, WT-78, WT-82, WT-88, WT-101, WT-105, WT-111, WT-115, WT-119 and WT-120. This is an incidental finding in mice.

The testes for WT-61, WT-62, WT-65, WT-68, WT-69, WT-70, WT-72, WT-74, WT-75, WT-78, WT-79. WT-81, WT-82, and WT-90 were microscopically normal. A few seminiferous tubules of animal ID WT-64, WT-80, WT-85, WT-88, and WT-89 had multiple variably sized vacuoles that replaced various levels of the seminiferous epithelium. There was no evidence of accompanying germ cell degeneration. Since there were a very few tubules affected and there was no accompanying degeneration, it suggests that this was an incidental finding. Animal ID WT-88 had a few mineralized seminiferous tubules. This dystrophic mineralization occurs where there is focal degeneration. Testicular degeneration occurs in approximately 20% of aged male mice and these lesions were most likely early signs of testicular degeneration in this mouse.

Ovaries were present for WT100, WT-104, WT-105, WT-112, WT115 and WT-119. All of the ovaries that were present were normal and the structures within the ovaries were consistent with various points in the estrus cycle.

The hearts and triceps muscles were microscopically normal in all of the animals.

The kidneys of mouse ID WT-49, WT-59, WT-60, WT-65, and WT-78 contained no microscopic abnormalities. The renal medulla had mild dilation of a few tubules in kidneys of WT-72, WT-90, WT-92, WT-95, WT-98, WT-99, WT-101, WT-102, WT-104, WT-112, WT-114, WT-115, WT-118, WT-119, and WT-120. The tubule dilation seen in these kidneys was mild simple dilation and was most likely due to a focal cellular cast. This lesion is not uncommon in mice.

The kidneys of WT-75, WT-62, WT-64, and WT-79 had mild focal interstitial fibrosis and infiltrates of lymphocytes and plasma cells. The kidney of WT-69 had focal interstitial fibrosis. There were mild perivascular infiltrates with small to moderate numbers of lymphocytes and plasma cells in kidneys of WT-61, WT-68, WT-70, WT-89, WT-WT-90, WT-91, WT-92, WT-95, WT-98, WT-99, WT-101, WT-102, WT-104, WT-112, WT-114, WT-115, WT-118, WT-119, and WT-120. The kidney of WT-111 had a few mineralized tubules, which mildly dilated the tubules. One-half of the kidney of WT-80 was contracted by interstitial fibrosis admixed with interstitial infiltrates with small numbers of lymphocytes and plasma cells. This caused hydronephrosis. Animals with ID WT-81, WT-82, WT-88, WT-100, and WT-105 contained mild multifocal interstitial and perivascular infiltrates with lymphocytes and plasma cells, interstitial fibrosis surrounding areas of tubular regeneration/regeneration. (Nephritis) The above-described lesions are considered incidental findings as they occur occasionally in adult or aged mice and typically are more frequent in male mice.

The livers of mouse ID WT-69, WT-70, WT-72, WT-75, WT-80, WT-99, WT-111, and WT-119 contained no microscopic abnormalities. The livers of WT-104, WT-105, WT-112, and WT-114 had mild to moderate perivascular infiltrates with lymphocytes and plasma cells with no corresponding fibrosis or hepatocellular necrosis. Mild perivascular infiltrates are a common finding in mice and increase in incidence as the mice age. Liver from WT-120 contained clusters of lymphoid hyperplasia. This was most likely a physiological response to gut related immune stimulation. This is considered an incidental finding.

The livers of WT-62, WT-64, WT-65, WT-68, WT-74, WT-79, WT-89, WT-91, WT-92, WT-95, WT-115, and WT-118 contained multifocal infiltrates with small numbers of mixed inflammatory cell infiltrates with hepatocellular necrosis (micro-abscess). Areas with 1-2 cell hepatocyte necrosis accompanied by inflammatory cells can occur spontaneously in the mouse liver with increased incidence as the mice age.

Animal numbers WT-82, WT-88, WT-98, WT-100, and WT-101 had multifocal areas of extramedullary hematopoiesis present in the liver parenchyma. This is less common in rodents as they age and typically occurs in response to increased hematopoietic demand.

Multifocal hepatocytes throughout the livers from WT-61, WT-78, WT-79, WT-81, WT-82, WT-85, WT-88, WT-89, and WT-90 had round variably sized intracytoplasmic vacuoles that are morphologically consistent with lipidosis. The lipidosis was mild and can occur as a normal finding depending on the metabolic status of the mouse.

The liver of WT-95 contained a focal area of hepatocellular hyperplasia (increased mitosis). This can occur as a spontaneous event or may have been due to prior liver damage.

The lungs of mouse ID WT-61, WT-64, WT-65, WT-68, WT-69, WT-70, WT-72, WT-74, WT-75, WT-78, WT-79, WT-80, WT-81, WT-82, WT-85, WT-88, WT-89, WT-90, WT-95, WT-98, WT-99, WT-100, WT-101, WT-102, WT-105, WT-111, WT-112, WT-114, WT-115, WT-118, and WT-120 contained no abnormalities. The lungs from the following mice had mild to moderate perivascular infiltrates with lymphocytes and plasma cells: WT-62, WT-91, WT-92, and WT-104. These inflammatory infiltrates are commonly seen in the lungs of adult mice.

The spleen of all mice had variable amounts of extramedullary hematopoiesis and hemosiderin within the macrophages of the red pulp. This is considered normal in older mice.

The tumors and increased number of inflammatory cell infiltrates and degenerative lesions seen in these mice are expected in mice as they age.

### Example 11: Conclusions from Safety and Toxicity Studies

IT AAV9/SLC52A2 doses up to 6×10¹¹ vg/mouse appears to be safe and well tolerated in WT mice. No outward signs of toxicity were noted over the duration of the study. Microscopic examination of major tissues/organs are ongoing.

What is claimed is:

1. A polynucleotide construct comprising a nucleic acid sequence comprising an engineered SLC52A2 gene sequence, or a cDNA thereof, or a variant thereof.

2. The polynucleotide construct of claim 1, wherein the polynucleotide construct further comprises one or more regulatory sequences operably linked to the nucleic acid sequence.

3. The polynucleotide construct of claim 1 or claim 2, wherein the one or more regulatory sequences comprise promoters, enhancers, polyadenylation signals, or terminators, or any combination thereof.

4. The polynucleotide construct of claim 3, wherein the promoter is a UsP promoter, or a variant thereof.

5. The polynucleotide construct of claim 3, wherein the one or more regulatory sequence comprise a UsP promoter and a polyadenylation signal.

6. The polynucleotide construct of any one of claims 1-5, wherein the nucleic acid sequence has a sequence as set forth in SEQ ID NO: 1, or a sequence at least about 60% identical thereto.

7. The polynucleotide construct of any one of claims 1-6, wherein the nucleic acid sequence encodes a Riboflavin Transporter 2 (RT), or any isoform thereof, or a variant thereof.

8. The polynucleotide construct of any one of claims 1-7, wherein the nucleic acid sequence encodes a polypeptide comprising an amino acid sequence as set forth in SEQ ID NO: 2, or an amino acid sequence at least about 60% identical thereto.

9. The polynucleotide construct of any one of claims 1-8, wherein the engineered SLC52A2 gene is codon optimized for expression in human.

10. The polynucleotide construct of claim 3, wherein the promoter comprises a nucleic acid sequence as set forth in SEQ ID NO: 3, or a sequence at least about 60% identical thereto.

11. A vector comprising a polynucleotide construct of any one of claims 1-10.

12. The vector of claim 11, wherein the vector is a viral vector.

13. A viral vector comprising a polynucleotide construct comprising a nucleic acid sequence comprising an engineered SLC52A2 gene sequence.

14. The viral vector of claim 13, wherein the viral vector is an adeno-associated viral vector.

15. The viral vector of claim 14, wherein the viral vector is an adeno-associated viral vector of serotype 9.

16. A delivery particle comprising the polynucleotide constructs of any one of claims 1-10, or the vector of any one of claims 11-12.

17. A transgenic cell comprising the polynucleotide construct sequence of any one of claims 1-10, or a vector of any one of claims 11-15.

18. A pharmaceutical composition comprising a polynucleotide construct of any one of claims 1-10, or a vector of any one of claims 11-15, or a transgenic cell of claim 17, and at least one pharmaceutically acceptable excipient.

19. A method of treating a disease or disorder in a subject in need thereof, the method comprising administering to the subject an effective amount of the pharmaceutical composition of claim 18.

20. The method of claim 19, wherein the subject has a defective SLC52A2 gene.

21. The method of claim 20, wherein the subject has or is suspected of having riboflavin transporter deficiency syndrome (RTD)

22. The method of claim 19 or claim 20, wherein the subject is a human.

23. A kit comprising the pharmaceutical composition comprising a polynucleotide construct of any one of claims 1-10, or a viral vector of any one of claims 13-15, or a transgenic cell of claim 17, and instructions for using the same.

24. We claim all methods, compositions, products and kits as shown and disclosed herein.


SEQUENCE LISTING (3 sequences)

SEQ ID NO: 1 - DNA, 1338 bp, synthetic construct
  source 1..1338: mol_type = other DNA
atggctgccccaactcccgcaagaccggtgttgacccatctcctcgtggccctgttcgggatgggatcatgggcggccgtgaatggcatttgggtggagctgcccgtggtggtcaaggagcttcccgagggatggtccctcccgtcctacgtgtccgtcctggtggccctgggaaacctgggcctcctggtggtcaccctgtggagaaggctcgcccctggaaaggatgaacaggtcccgattcgcgtggtccaggtcctggggatggtcggcaccgctctgctggcctccctgtggcaccacgtggcaccagtcgcgggacagctgcactccgtggcgttcttagcccttgccttcgtgctcgccttggcctgctgtgcctcgaacgtgacgttcctgcccttcctgtcccatcttcccccccggtttctgcggtcgttcttcctgggacagggactgagcgccctgctgccttgtgtgctggcccttgtgcaaggagtgggacgcctggaatgtccgccggcacccatcaacgggactccaggccctcctctggacttcctggaacggttcccggcctccacctttttctgggcgctgacagcgttgcttgtggcgtcagctgcagcgtttcagggccttctgctcctcctccctcctccgccttccgtgccgactggggaactgggctctgggctccaagtcggagccccaggagctgaggaagaagtggaggaatcctcgccgttgcaagagcccccgtcacaagcggctggcaccactcccggtcctgaccccaaagcataccagctgctgtcggctaggagcgcctgtctgctgggactcctcgctgcgactaacgcgctgaccaacggagtgctcccagccgtgcagtccttctcctgcctgccgtacggtcgcctggcctaccacctggccgtggtgctgggtagcgccgctaaccctctggcctgcttcctggccatgggcgtgttgtgccggtcactggctgggctgggaggacttagcctcctgggagtgttctgcggtggctacctgatggccctggccgtcttgtccccctgccctcctctcgtgggaacctccgccggagtggtcttggtggtgctgtcgtgggtgctgtgtctcggggtgttcagctacgtgaaggttgccgcgtcaagcctgctccacggtggcggaaggcctgcactgctggcagcaggcgtagccatccaagtcggcagcctactcggagccgtggccatgttcccgccgacctccatctatcacgtctttcattcccggaaggactgcgccgatccatgcgacagctga

SEQ ID NO: 2 - protein, 445 aa, synthetic construct
MAAPTPARPVLTHLLVALFGMGSWAAVNGIWVELPVVVKELPEGWSLPSYVSVLVALGNLGLLVVTLWRRLAPGKDEQVPIRVVQVLGMVGTALLASLWHHVAPVAGQLHSVAFLALAFVLALACCASNVTFLPFLSHLPPRFLRSFFLGQGLSALLPCVLALVQGVGRLECPPAPINGTPGPPLDFLERFPASTFFWALTALLVASAAAFQGLLLLLPPPPSVPTGELGSGLQVGAPGAEEEVEESSPLQEPPSQAAGTTPGPDPKAYQLLSARSACLLGLLAATNALTNGVLPAVQSFSCLPYGRLAYHLAVVLGSAANPLACFLAMGVLCRSLAGLGGLSLLGVFCGGYLMALAVLSPCPPLVGTSAGVVLVVLSWVLCLGVFSYVKVAASSLLHGGGRPALLAAGVAIQVGSLLGAVAMFPPTSIYHVFHSRKDCADPCDS

SEQ ID NO: 3 - DNA, 320 bp, synthetic construct
  source 1..320: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag